(19) United States
(12) Patent Application Publication
(10) Pub. No.: US 2014/0350228 A1
(43) Pub. Date: Nov. 27, 2014

(54) USE OF N-HYDROXYSUCCINIMIDE TO IMPROVE CONJUGATE STABILITY

(71) Applicant: ImmunoGen, Inc., Waltham, MA (US)

(72) Inventors: Fang Liu, Lexington, MA (US); Godfrey W. Amphlett, Cambridge, MA (US); Deborah H. Meshulam, Brookline, MA (US)

(73) Assignee: ImmunoGen, Inc., Waltham, MA (US)

(21) Appl. No.: 14/365,305

(22) PCT Filed: Dec. 13, 2012
(86) PCT No.: PCT/US12/69527

§ 371 Date: Jun. 13, 2014

Related U.S. Application Data
(60) Provisional application No. 61/570,139, filed on Dec. 13, 2011.

(51) Int. Cl. A61K 47/48

(52) U.S. Cl. CPC A61K 47/48384
USPC 530/391.7

(57) ABSTRACT

The invention provides processes for manufacturing cell-binding agent-cytotoxic agent conjugates of improved stability in the presence of exogenous NHS. In some embodiments, the inventive process comprises the addition of a molar ratio of exogenous NHS with respect to the amount of NHS generated during the modification reaction as a result of hydrolysis/aminolysis of the bifunctional linker.

### CROSS-REFERENCE TO RELATED APPLICATIONS

This patent application claims the benefit of U.S. Provisional Patent Application No. 61/570,139, filed Dec. 13, 2011, which is incorporated by reference.

### BACKGROUND OF THE INVENTION

Antibody-drug conjugates which are useful for the treatment of cancer and other diseases are commonly composed of three distinct elements: a cell-binding agent; a linker; and a cytotoxic agent. One of the commonly used manufacturing processes comprises a modification step, in which the cell-binding agent is reacted with a bifunctional linker to form a cell-binding agent covalently attached to a linker having a reactive group; a purification step, in which the modified antibody is purified from the other components of the modification reaction; a conjugation step, in which the modified cell-binding agent is reacted with a cytotoxic agent to form a covalent chemical bond from the linker (using the reactive group) to the cytotoxic agent; and a second purification step, in which the conjugate is purified from the other components of the conjugation reaction.

Despite advances in preparing antibody-drug conjugates, current processes are limited by several factors. For example, the binding of a bifunctional cross-linking agent to an antibody is heterogeneous under the conditions currently employed in the art, resulting in a conjugate comprising stable amide bonds and unstable ester bonds. It is thought that the presence of unstable ester bonds in the conjugate lead to the slow release of the drug from the conjugate and conjugate instability.

Recent clinical trials have shown a promising role for antibody-drug conjugates in the treatment of many different types of cancers. Thus, there remains a need for improved processes of preparing antibody-drug conjugates that are more stable and are of higher purity than antibody-drug conjugates produced by current processes. The invention provides such a process. These and other advantages of the invention, as well as additional inventive features, will be apparent from the description of the invention provided herein.

### BRIEF SUMMARY OF THE INVENTION

The invention provides processes for manufacturing cell-binding agent-cytotoxic agent conjugates of improved stability in the presence of exogenous N-hydroxysuccinimide (NHS).

### DETAILED DESCRIPTION OF THE INVENTION

One of ordinary skill in the art will appreciate that conjugates comprising an antibody chemically coupled to a cytotoxic agent (“antibody-cytotoxic agent conjugates”) typically are prepared by modifying an antibody with a bifunctional crosslinking reagent, often utilizing an N-hydroxysuccinimide (NHS) reactive group on the crosslinking agent, purifying the antibody having linkers bound thereto, conjugating a cytotoxic agent to the antibody having linkers bound thereto, and purifying the antibody-cytotoxic agent conjugate. The invention improves upon such methods by maximizing the amount of linker stably bound to the cell-binding agent and minimizing undesirable side reactions that lead to conjugate instability.

A small amount of NHS is generated during the modification reaction as a result of hydrolysis/aminolysis of the bifunctional linker (e.g., SPP, SPDB, SMCC). NHS currently is regarded by those of skill in the art as an undesirable (or at best neutral) byproduct of the modification reaction. Therefore, current methods typically include purification of the modified antibody prior to addition of cytotoxic agent, which results in removal of NHS prior to the conjugation reaction.

It was surprisingly discovered that preparing antibody-cytotoxic agent conjugates in the presence of exogenous NHS results in a significant increase in the stability of the conjugate, as measured by release of free maytansinoid. Accordingly, the invention provides processes for manufacturing cell-binding agent-cytotoxic agent conjugates of improved stability in the presence of exogenous NHS.

The invention provides a process for preparing a cell-binding agent-cytotoxic agent conjugate, which process comprises the addition of exogenous NHS. “Exogenous NHS,” as used herein, refers to NHS that is added during the process from an external source, and does not refer to NHS that is generated during the modification reaction as a result of hydrolysis/aminolysis of the bifunctional linker.

In one embodiment, the invention provides a process for preparing a cell-binding agent-cytotoxic agent conjugate, which process comprises the addition of about 0.1 mM to about 300 mM exogenous NHS. For example, the inventive process comprises the addition of about 0.1 mM, about 0.2 mM, about 0.3 mM, about 0.4 mM, about 0.5 mM, about 0.6 mM, about 0.7 mM, about 0.8 mM, about 0.9 mM, about 1.0 mM, about 1.1 mM, about 1.3 mM, about 1.5 mM, about 1.7 mM, about 1.9 mM, about 2.0 mM, about 2.1 mM, about 2.3 mM, about 2.5 mM, about 2.7 mM, about 2.9 mM, about 3.0 mM, about 3.1 mM, about 3.3 mM, about 3.5 mM, about 3.7 mM, about 3.9 mM, about 4.0 mM, about 4.1 mM, about 4.3 mM, about 4.5 mM, about 4.7 mM, about 4.9 mM, about 5.0 mM, about 5.1 mM, about 5.3 mM, about 5.5 mM, about 5.7 mM, about 5.9 mM, about 6.0 mM, about 6.1 mM, about 6.3 mM, about 6.5 mM, about 6.7 mM, about 6.9 mM, about 7.0 mM, about 7.1 mM, about 7.3 mM, about 7.5 mM, about 7.7 mM, about 7.9 mM, about 8.0 mM, about 8.1 mM, about 8.3 mM, about 8.5 mM, about 8.7 mM, about 8.9 mM, about 9.0 mM, about 9.1 mM, about 9.3 mM, about 9.5 mM, about 9.7 mM, about 9.9 mM, about 10 mM, about 11 mM, about 12 mM, about 13 mM, about 14 mM, about 15 mM, about 16 mM, about 17 mM, about 18 mM, about 19 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, or about 300 mM exogenous NHS. In one embodiment, the inventive process comprises the addition of about 0.1 mM to about 5 mM, about 0.1 mM to about 10 mM, about 1.0 mM to about 5 mM, about 1.0 mM to about 10 mM, about 5.0 mM to about 10 mM, about 10 mM to about 20 mM, about 20 mM to about 30 mM, about 30 mM to about 40 mM, about 40 mM to about 50 mM, about 50 mM to about 60 mM, about 60 mM to about 70 mM, about 70 mM to about 80 mM, about 80 mM to about 90 mM, about 90 mM to about 100 mM, about 100 mM to about 110 mM, about 110 mM to about 120 mM, about 120 mM to about 130 mM, about 130 mM to about 140 mM, about 140 mM to about 150 mM, about 150 mM to about 160 mM, about 160 mM to about 170 mM, about 170 mM to about 180 mM, about 180 mM to about 190 mM, about 190 mM to about 200 mM, about 200 mM to about 220 mM, about 220 mM to about 240 mM, about 240 mM to about 260 mM, about 260 mM to about 280 mM, or about 280 mM to about 300 mM exogenous NHS. In another embodiment, the inventive process comprises the addition of about 10 mM to about 200 mM, about 20 to about 150 mM, about 50 to about 150 mM, or about 20 to about 100 mM exogenous NHS.

In some embodiments, the inventive process comprises the addition of a molar ratio of exogenous NHS with respect to the amount of NHS generated during the modification reaction as a result of hydrolysis/aminolysis of the bifunctional linker. One of ordinary skill in the art can determine the amount of NHS generated during a particular modification as the amount of NHS generated is essentially the same as the amount of the bifunctional linker used. The skilled person can then add a molar ratio of exogenous NHS to the reaction mixture with respect to the amount of NHS generated during the modification reaction. In one embodiment, about 2 to about 200 fold exogenous NHS is added with respect to the amount of NHS generated during the modification reaction. For example, the inventive process comprises adding about 2, about 5, about 10, about 15, about 20, about 25, about 50, about 100, or about 200 fold exogenous NHS with respect to the amount of NHS generated during the modification reaction.

In some embodiments, the inventive process comprises the addition of a molar ratio of exogenous NHS with respect to the amount of the bifunctional linker. In one embodiment, the molar ratio of the exogenous NHS to the bifunctional crosslinking agent is about 0.5 to about 1000 (e.g., about 1 to about 900, about 5 to about 750, about 50 to about 500, about 100 to about 500, about 0.5 to about 500, or about 100 to about 1000). For example, the inventive process comprises about 0.5, about 1, about 2, about 5, about 10, about 15, about 20, about 25, about 50, about 100, about 200, about 300, about 400, about 500, about 600, about 700, about 800, about 900, or about 1000 fold NHS with respect to the amount of the bifunctional linker.

A number of processes for preparing cell-binding agent-cytotoxic agent conjugates have been described (see, e.g., U.S. Provisional Patent Application No. 61/468,997, U.S. Pat. No. 5,208,020, U.S. Pat. No. 6,441,163, U.S. Pat. No. 7,811,572, U.S. Patent Application Publication No. 2006/0182750, U.S. Patent Application Publication No. 2008/0145374, U.S. Patent Application Publication No. 2011/0003969, and U.S. Patent Application Publication No. 2012/0253021). Applicants have surprisingly discovered that the addition of exogenous NHS at any point during a process for preparing a cell-binding agent-cytotoxic agent conjugate has a beneficial effect on conjugate stability. Thus, the inventive process comprises the addition of exogenous NHS at any point during a process preparing a cell-binding agent-cytotoxic agent conjugate. For example, the inventive process comprises the addition of exogenous NHS to the modification step (i.e., the step in which a cell-binding agent is reacted with a bifunctional linker), to the conjugation step (i.e., the step in which a modified cell-binding agent is reacted with a cytotoxic agent), to a purification step, or to a holding step between any of the foregoing steps. In one embodiment, the inventive process comprises the addition of exogenous NHS to the modification step (i.e., NHS is added to the modification reaction), to a holding step between the modification step and a purification step, to a holding step between the modification step and the conjugation step, to a purification step, to the conjugation step, to a holding step between the conjugation step and a purification step, and/or to a holding step between two purification steps.

In one embodiment, the invention provides a process for preparing a cell-binding agent having a linker bound thereto, which process comprises contacting a cell-binding agent with a bifunctional crosslinking reagent in the presence of exogenous NHS to covalently attach a linker to the cell-binding agent and thereby prepare a mixture comprising cell-binding agents having linkers bound thereto.

In accordance with the inventive method, contacting a cell-binding agent with a bifunctional crosslinking reagent (i.e., the modification reaction) produces a first mixture comprising the cell-binding agent having linkers bound thereto, as well as reactants and other by-products. In some embodiments of the invention, the first mixture comprises the cell-binding agent having linkers stably and unstably bound thereto, as well as reactants and other by-products. A linker is “stably” bound to the cell-binding agent when the covalent bond between the linker and the cell-binding agent is not substantially weakened or severed under normal storage conditions over a period of time, which could range from a few months to a few years. In contrast, a linker is “unstably” bound to the cell-binding agent when the covalent bond between the linker and the cell-binding agent is substantially weakened or severed under normal storage conditions over a period of time, which could range from a few months to a few years.

The modification reaction preferably is performed at a pH of about 4 to about pH 9 (e.g., a pH of about 4.5 to about 8.5, about 5 to about 8, about 5.5 to about 7.5, about 6 to about 7, about 6 to about 8, about 6 to about 9, or about 6.5 to about 7.5). In some embodiments, the modification reaction is performed at a pH of about 6 to about 8 (e.g., a pH of about 6, about 6.5, about 7, about 7.5, or about 8).

In one embodiment of the invention, purification of the modified cell-binding agent from reactants and by-products is carried out by subjecting the first mixture to a purification process. In this regard, the first mixture can be purified using tangential flow filtration (TFF), e.g., a membrane-based tangential flow filtration process, non-adsorptive chromatography, adsorptive chromatography, adsorptive filtration, or selective precipitation, or any other suitable purification process, as well as combinations thereof. This first purification step provides a purified first mixture, i.e., an increased concentration of the cell-binding agents having linkers bound thereto and a decreased amount of unbound bifunctional crosslinking reagent, as compared to the first mixture prior to purification in accordance with the invention. Preferably, the first mixture is purified using tangential flow filtration.

After purification of the first mixture to obtain a purified first mixture of cell-binding agents having linkers bound thereto, a cytotoxic agent is conjugated to the cell-binding agents having linkers bound thereto in the first purified mixture by reacting the cell-binding agents having linkers bound thereto with a cytotoxic agent in a solution having a pH from about 4 to about 9 to form a second mixture, wherein a second mixture comprising (i) the cell-binding agent chemically coupled through the linker to the cytotoxic agent, (ii) free cytotoxic agent, and (iii) reaction by-products is produced.

Optionally, purification of the modified cell-binding agent may be omitted. Thus, in one embodiment of the invention, the first mixture comprising the cell-binding agent having linkers bound thereto, as well as reactants and other by-products, is not subjected to a purification process. In such a situation, the cytotoxic agent may be added simultaneously with the crosslinking reagent or at some later point, e.g., 1, 2, 3, or more hours after addition of the crosslinking reagent to the cell-binding agent. The modified cell-binding agent is conjugated to a cytotoxic agent (e.g., a maytansinoid) by reacting the modified cell-binding agent with the cytotoxic agent in a solution having a pH from about 4 to about 9, wherein the conjugation step results in formation of a mixture of stable cell-binding agent-cytotoxic agent conjugates, non-stable cell-binding agent-cytotoxic agent conjugates, non-conjugated cytotoxic agent (i.e., “free” cytotoxic agent), reactants, and by-products.

The conjugation reaction preferably is performed at a pH of about 4 to about pH 9 (e.g., a pH of about 4.5 to about 8.5, about 5 to about 8, about 5.5 to about 7.5, about 6.0 to about 7, or about 6.5 to about 7.5). In some embodiments, the conjugation reaction is performed at a pH of about 6 to about 6.5 (e.g., a pH of 5.5 to 7, a pH of 5.7 to 6.8, a pH of 5.8 to 6.7, a pH of 5.9 to 6.6, or a pH of 6 to 6.5), a pH of about 6 or below (e.g., a pH of about 4 to 6, about 4 to about 5.5, about 5 to 6) or at a pH of about 6.5 or greater (e.g., a pH of 6.5 to about 9, about 6.5 to about 7, about 7 to about 9, about 7.5 to about 9, or 6.5 to about 8). In one embodiment, the conjugation reaction is performed at a pH of about 4 to a pH less than 6 or at a pH of greater than 6.5 to 9. When the conjugation step is performed at a pH of about 6.5 or greater, some sulfhydryl-containing cytotoxic agents may be prone to dimerize by disulfide-bond formation. In one embodiment, removal of trace metals and/or oxygen from the reaction mixture, as well as optional addition of antioxidants or the use of linkers with more reactive leaving groups, or addition of cytotoxic agent in more than one aliquot, may be required to allow for efficient reaction in such a situation.

The inventive process may optionally include the addition of sucrose to the conjugation step used in the inventive process to increase solubility and recovery of the cell-binding agent-cytotoxic agent conjugates. Desirably, sucrose is added at a concentration of about 0.1% (w/v) to about 20% (w/v) (e.g., about 0.1% (w/v), 1% (w/v), 5% (w/v), 10% (w/v), 15% (w/v), or 20% (w/v)). Preferably, sucrose is added at a concentration of about 1% (w/v) to 10% (w/v) (e.g., (e.g., about 0.5% (w/v), about 1% (w/v), about 1.5% (w/v), about 2% (w/v), about 3% (w/v), about 4% (w/v), about 5% (w/v), about 6% (w/v), about 7% (w/v), about 8% (w/v), about 9% (w/v), about 10% (w/v), or about 11% (w/v)). In addition, the conjugation reaction also can comprise the addition of a buffering agent. Any suitable buffering agent known in the art can be used. Suitable buffering agents include, for example, a citrate buffer, an acetate buffer, a succinate buffer, and a phosphate buffer. In a preferred embodiment, the buffering agent is selected from the group consisting of HEPPSO (N-(2-Hydroxyethyl)piperazine-N′-(2-hydroxypropanesulfonic acid)), POPSO (Piperazine-1,4-bis-(2-hydroxy-propane-sulfonic acid) dehydrate), HEPES (4-(2-hydroxyethyl)piperazine-1-ethanesulfonic acid), HEPPS (EPPS) (4-(2-hydroxyethyl)piperazine-1-propanesulfonic acid), TES (N-[tris(hydroxymethyl)methyl]-2-aminoethanesulfonic acid), and a combination thereof.

Following the conjugation step, the conjugate is subjected to a purification step. In this regard, the conjugation mixture can be purified using tangential flow filtration (TFF), e.g., a membrane-based tangential flow filtration process, non-adsorptive chromatography, adsorptive chromatography, adsorptive filtration, or selective precipitation, or any other suitable purification process, as well as combinations thereof. One of ordinary skill in the art will appreciate that purification after the conjugation step enables the isolation of a stable conjugate comprising the cell-binding agent chemically coupled to the cytotoxic agent.

In one embodiment, the invention provides a process for preparing a conjugate comprising a cell-binding agent chemically coupled to a cytotoxic agent, which process comprises a first purification step after the modification step and a second purification step after the conjugation step, wherein the process comprises exogenous NHS. Exogenous NHS can be added at any point during the inventive process (i.e., to the modification step, to the conjugation step, to the purification step(s), or to a holding step(s) between any one of the aforementioned steps). For example, in one embodiment, the invention provides a process for preparing a cell-binding agent-cytotoxic agent conjugate comprising a cell-binding agent chemically coupled to a cytotoxic agent through a linker, which process comprises (a) contacting a cell-binding agent with a bifunctional crosslinking reagent to covalently attach a linker to the cell-binding agent and thereby prepare a first mixture comprising cell-binding agents having linkers bound thereto, (b) subjecting the first mixture to tangential flow filtration, selective precipitation, non-adsorptive chromatography, adsorptive filtration, adsorptive chromatography, or a combination thereof and thereby prepare a purified first mixture of cell-binding agents having linkers bound thereto, (c) conjugating a cytotoxic agent to the cell-binding agents having linkers bound thereto in the purified first mixture by reacting the cell-binding agents having linkers bound thereto with a cytotoxic agent to prepare a second mixture comprising (i) the cell-binding agent-cytotoxic agent conjugate comprising the cell-binding agent chemically coupled to the cytotoxic agent through the linker, (ii) free cytotoxic agent, and (iii) reaction by-products, and (d) subjecting the second mixture to tangential flow filtration, selective precipitation, non-adsorptive chromatography, adsorptive filtration, adsorptive chromatography, or a combination thereof to purify the cell-binding agent-cytotoxic agent conjugate from the other components of the second mixture and thereby prepare a purified second mixture of the cell-binding agent-cytotoxic agent conjugate, wherein exogenous NHS is added during or after step (a) and prior to step (c) (i.e., the contacting in step (a) is carried out in the presence of exogenous NHS, the process comprises holding the first mixture after step (a) in the presence of exogenous NHS, exogenous NHS is added in step (b), and/or the process comprises holding the first mixture after step (b) in the presence of exogenous NHS). In another embodiment, the invention provides a process for preparing a cell-binding agent-cytotoxic agent conjugate comprising a cell-binding agent chemically coupled to a cytotoxic agent through a linker, which process comprises (a) contacting a cell-binding agent with a bifunctional crosslinking reagent to covalently attach a linker to the cell-binding agent and thereby prepare a first mixture comprising cell-binding agents having linkers bound thereto, (b) subjecting the first mixture to tangential flow filtration, selective precipitation, non-adsorptive chromatography, adsorptive filtration, adsorptive chromatography, or a combination thereof and thereby prepare a purified first mixture of cell-binding agents having linkers bound thereto, (c) conjugating a cytotoxic agent to the cell-binding agents having linkers bound thereto in the purified first mixture by reacting the cell-binding agents having linkers bound thereto with a cytotoxic agent in the presence of exogenous N-hydroxysuccinimide to prepare a second mixture comprising (i) the cell-binding agent-cytotoxic agent conjugate comprising the cell-binding agent coupled to the cytotoxic agent through the linker, (ii) free cytotoxic agent, and (iii) reaction by-products, and (d) subjecting the second mixture to tangential flow filtration, selective precipitation, non-adsorptive chromatography, adsorptive filtration, adsorptive chromatography, or a combination thereof to purify the cell-binding agent-cytotoxic agent conjugate from the other components of the second mixture and thereby prepare a purified second mixture of cell-binding agent-cytotoxic agent conjugate. In yet another embodiment, the invention provides a process for preparing a cell-binding agent-cytotoxic agent conjugate comprising a cell-binding agent chemically coupled to a cytotoxic agent through a linker, which process comprises (a) contacting a cell-binding agent with a bifunctional crosslinking reagent to covalently attach a linker to the cell-binding agent and thereby prepare a first mixture comprising cell-binding agents having linkers bound thereto, (b) subjecting the first mixture to tangential flow filtration, selective precipitation, non-adsorptive chromatography, adsorptive filtration, adsorptive chromatography, or a combination thereof and thereby prepare a purified first mixture of cell-binding agents having linkers bound thereto, (c) conjugating a cytotoxic agent to the cell-binding agents having linkers bound thereto in the purified first mixture by reacting the cell-binding agents having linkers bound thereto with a cytotoxic agent to prepare a second mixture comprising (i) the cell-binding agent-cytotoxic agent conjugate comprising the cell-binding agent chemically coupled through the linker to the cytotoxic agent, (ii) free cytotoxic agent, and (iii) reaction by-products, (d) incubating the second mixture in the presence of exogenous N-hydroxysuccinimide; and (e) subjecting the second mixture after step (d) to tangential flow filtration, selective precipitation, non-adsorptive chromatography, adsorptive filtration, adsorptive chromatography, or a combination thereof to purify the cell-binding agent-cytotoxic agent conjugate from the other components of the second mixture and thereby prepare a purified second mixture of cell-binding agents chemically coupled through the linkers to the cytotoxic agent.

Any purification method described herein can be used in the inventive process. In one embodiment of the invention, tangential flow filtration (TFF, also known as cross flow filtration, ultrafiltration and diafiltration) and/or adsorptive chromatography resins are utilized in the purification steps. For example, the inventive process can comprise a first purification step using TFF after the modification step and a second purification step using TFF after the conjugation step. Alternatively, the inventive process can comprise a first purification step using adsorptive chromatography after the modification step and a second purification step using adsorptive chromatography after the conjugation step. The inventive process also can comprise a first purification step using adsorptive chromatography after the modification step and a second purification step using TFF after the conjugation step or a first purification step using TFF after the modification step and a second purification step using adsorptive chromatography after the conjugation step.

In one embodiment of the invention, non-adsorptive chromatography is utilized as the purification step. For example, the inventive process can comprise a first purification step using non-adsorptive chromatography after the modification step and a second purification step using non-adsorptive chromatography after the conjugation step.

In another embodiment, the invention provides a process for preparing a conjugate comprising a cell-binding agent chemically coupled to a cytotoxic agent, wherein the first mixture comprising cell-binding agents having linkers bound thereto is not subjected to purification following the modification reaction and prior to the conjugation reaction, and wherein the process comprises exogenous NHS. Exogenous NHS can be added at any point during the inventive process (i.e., to the modification step, to the conjugation step, to the purification step(s), or to a holding step(s) between any one of the aforementioned steps). Thus, in one embodiment, the invention provides a process for preparing a cell-binding agent-cytotoxic agent conjugate comprising a cell-binding agent chemically coupled to a cytotoxic agent through a linker, which process comprises (a) contacting a cell-binding agent with a bifunctional crosslinking reagent to covalently attach a linker to the cell-binding agent and thereby prepare a first mixture comprising cell-binding agents having linkers bound thereto, (b) conjugating a cytotoxic agent to the cell-binding agents having linkers bound thereto in the first mixture by reacting the cell-binding agents having linkers bound thereto with a cytotoxic agent to prepare a second mixture comprising (i) the cell-binding agent-cytotoxic agent conjugate comprising the cell-binding agent coupled to the cytotoxic agent through the linker, (ii) free cytotoxic agent, and (iii) reaction by-products, and (c) subjecting the second mixture to tangential flow filtration, selective precipitation, non-adsorptive chromatography, adsorptive filtration, adsorptive chromatography, or a combination thereof, to purify the cell-binding agent-cytotoxic agent conjugate from the other components of the second mixture and thereby prepare a purified second mixture of the cell-binding agent-cytotoxic agent conjugate, wherein exogenous N-hydroxysuccinimide is added during or after step (a) and prior to step (b) (i.e., the contacting in step (a) is carried out in the presence of exogenous NHS and/or the process comprises holding the first mixture after step (a) in the presence of exogenous NHS). In another embodiment, the invention provides a process for preparing a cell-binding agent-cytotoxic agent conjugate comprising a cell-binding agent chemically coupled to a cytotoxic agent through a linker, which process comprises (a) contacting a cell-binding agent with a bifunctional crosslinking reagent to covalently attach a linker to the cell-binding agent and thereby prepare a first mixture comprising cell-binding agents having linkers bound thereto, (b) conjugating a cytotoxic agent to the cell-binding agents having linkers bound thereto in the first mixture by reacting the cell-binding agents having linkers bound thereto with a cytotoxic agent in the presence of exogenous N-hydroxysuccinimide to prepare a second mixture comprising (i) the cell-binding agent-cytotoxic agent conjugate comprising cell-binding agent chemically coupled through the linker to the cytotoxic agent, (ii) free cytotoxic agent, and (iii) reaction by-products, and (c) subjecting the second mixture to tangential flow filtration, selective precipitation, non-adsorptive chromatography, adsorptive filtration, adsorptive chromatography, or a combination thereof, to purify the cell-binding agent-cytotoxic agent conjugate from the other components of the second mixture and thereby prepare a purified second mixture of the cell-binding agent-cytotoxic agent conjugate. In yet another embodiment, the invention provides a process for preparing a conjugate comprising a cell-binding agent chemically coupled to a cytotoxic agent through a linker, which process comprises (a) contacting a cell-binding agent with a bifunctional crosslinking reagent to covalently attach a linker to the cell-binding agent and thereby prepare a first mixture comprising cell-binding agents having linkers bound thereto, (b) conjugating a cytotoxic agent to the cell-binding agents having linkers bound thereto in the first mixture by reacting the cell-binding agents having linkers bound thereto with a cytotoxic agent to prepare a second mixture comprising (i) the cell-binding agent-cytotoxic agent conjugate comprising the cell-binding agent chemically coupled through the linker to the cytotoxic agent, (ii) free cytotoxic agent, and (iii) reaction by-products, (c) incubating the second mixture in the presence of exogenous N-hydroxysuccinimide; and (d) subjecting the second mixture after step (c) to tangential flow filtration, selective precipitation, non-adsorptive chromatography, adsorptive filtration, adsorptive chromatography, or a combination thereof, to purify the cell-binding agent-cytotoxic agent conjugate from the other components of the second mixture and thereby prepare a purified second mixture of the cell-binding agent-cytotoxic agent conjugate. Any purification method described herein can be used as the purification step following the conjugation reaction. In a preferred embodiment, tangential flow filtration, adsorptive chromatography, or non-adsorptive chromatography is utilized as the purification step following the conjugation reaction.

In another embodiment, the invention provides a process for preparing a conjugate comprising a cell-binding agent chemically coupled to a cytotoxic agent, wherein the modification reaction and the conjugation reaction are combined into a single step, followed by a purification step (as described in U.S. Provisional Patent Application No. 61/468,997 and U.S. Patent Application Publication No. 2012/0253021), and wherein the process comprises exogenous NHS. Exogenous NHS can be added at any point during the inventive process (i.e., to the combined modification/conjugation step, to the purification step, or to a holding step between the aforementioned steps). Thus, in one embodiment, the invention provides a process for preparing a cell-binding agent-cytotoxic agent conjugate comprising a cell-binding agent chemically coupled to a cytotoxic agent through a linker, which process comprises (a) contacting a cell-binding agent with a cytotoxic agent in the presence of exogenous N-hydroxysuccinimide to form a first mixture comprising the cell-binding agent and the cytotoxic agent, then contacting the first mixture with a bifunctional crosslinking reagent comprising a linker, in a solution having a pH of about 4 to about 9, to provide a second mixture comprising (i) the cell-binding agent cytotoxic agent conjugate comprising the cell-binding agent chemically coupled through the linker to the cytotoxic agent, (ii) free cytotoxic agent, and (iii) reaction by-products; and (b) subjecting the second mixture to tangential flow filtration, selective precipitation, non-adsorptive chromatography, adsorptive filtration, adsorptive chromatography, or a combination thereof, to purify the cell-binding agent-cytotoxic agent conjugate from the other components of the second mixture and thereby prepare a purified second mixture of the cell-binding agent-cytotoxic agent conjugate. In another embodiment, the invention provides a process for preparing a cell-binding agent-cytotoxic agent conjugate comprising a cell-binding agent chemically coupled to a cytotoxic agent through a linker, which process comprises (a) contacting a cell-binding agent with a cytotoxic agent to form a first mixture comprising the cell-binding agent and the cytotoxic agent, then contacting the first mixture with a bifunctional crosslinking reagent comprising a linker in the presence of exogenous N-hydroxysuccinimide, in a solution having a pH of about 4 to about 9, to provide a second mixture comprising (i) the cell-binding agent cytotoxic agent conjugate comprising the cell-binding agent chemically coupled through the linker to the cytotoxic agent, (ii) free cytotoxic agent, and (iii) reaction by-products; and (b) subjecting the second mixture to tangential flow filtration, selective precipitation, non-adsorptive chromatography, adsorptive filtration, adsorptive chromatography, or a combination thereof, to purify the cell-binding agent-cytotoxic agent conjugate from the other components of the second mixture and thereby prepare a purified second mixture of the cell-binding agent-cytotoxic agent conjugate. In another embodiment, the invention provides a process for preparing a cell-binding agent-cytotoxic agent conjugate comprising a cell-binding agent chemically coupled to a cytotoxic agent through a linker, which process comprises (a) contacting a cell-binding agent with a cytotoxic agent to form a first mixture comprising the cell-binding agent and the cytotoxic agent, then contacting the first mixture with a bifunctional crosslinking reagent comprising a linker, in a solution having a pH of about 4 to about 9, to provide a second mixture comprising (i) the cell-binding agent cytotoxic agent conjugate comprising the cell-binding agent chemically coupled through the linker to the cytotoxic agent, (ii) free cytotoxic agent, and (iii) reaction by-products; (b) incubating the second mixture in the presence of exogenous N-hydroxysuccinimide; and (c) subjecting the second mixture after step (b) to tangential flow filtration, selective precipitation, non-adsorptive chromatography, adsorptive filtration, adsorptive chromatography, or a combination thereof, to purify the cell-binding agent-cytotoxic agent conjugate from the other components of the second mixture and thereby prepare a purified second mixture of the cell-binding agent-cytotoxic agent conjugate. In one embodiment, the incubating of step (b) (i.e., the holding step following the combined modification/conjugation step) is carried out immediately after the first mixture is contacted with the bifunctional crosslinking reagent. In one embodiment, the process comprises subjecting the second mixture to purification between steps (a)-(b) to purify the cell-binding agent-cytotoxic agent conjugate from the other components of the second mixture and thereby prepare a purified second mixture of the cell-binding agent-cytotoxic agent conjugate prior to the holding step (i.e., prior to step (b)). Any purification method described herein can be used in the inventive process. In a preferred embodiment, tangential flow filtration, adsorptive chromatography, or non-adsorptive chromatography is utilized as the purification step.

In one embodiment, the invention provides a process for preparing a conjugate comprising a cell-binding agent chemically coupled to a cytotoxic agent, wherein the process comprises conjugating a pre-formed cytotoxic-agent-linker compound to a cell-binding agent, as described in U.S. Pat. No. 6,441,163 and U.S. Patent Application Publication Nos. 2011/0003969 and 2008/0145374, and wherein the process comprises exogenous NHS. Exogenous NHS can be added at any point during the inventive process (i.e., to the conjugation step, to the purification step, or to a holding step between the aforementioned steps). For example, in one embodiment, the invention provides a process for preparing a cell-binding agent-cytotoxic agent conjugate comprising a cell-binding agent chemically coupled to a cytotoxic agent through a linker, which process comprises (a) contacting a cell-binding agent with a cytotoxic agent-linker compound comprising a cytotoxic agent chemically coupled to a linker to covalently attach the cytotoxic agent-linker compound to the cell-binding agent and thereby prepare a mixture comprising the cell-binding agent-cytotoxic agent conjugate comprising the cell-binding agent chemically coupled to the cytotoxic agent through the linker and (b) subjecting the mixture comprising the cell-binding agent-cytotoxic agent conjugate to tangential flow filtration, selective precipitation, non-adsorptive chromatography, adsorptive filtration, adsorptive chromatography or a combination thereof to purify the conjugate, wherein exogenous N-hydroxysuccinimide is added during or after step (a) and prior to step (b) (i.e., the contacting in step (a) is carried out in the presence of exogenous NHS or the process comprises holding the mixture after (a) in the presence of exogenous NHS). In another embodiment, the invention provides a process for preparing a cell-binding agent-cytotoxic agent conjugate comprising a cell-binding agent chemically coupled to a cytotoxic agent through a linker, which process comprises (a) contacting a cell-binding agent with a cytotoxic agent-linker compound comprising a cytotoxic agent chemically coupled to a linker to covalently attach the cytotoxic agent-linker compound to the cell-binding agent and thereby prepare a mixture comprising the cell-binding agent-cytotoxic agent conjugate comprising the cell-binding agent chemically coupled to the cytotoxic agent through the linker; (b) incubating the mixture of step (a) in the presence of exogenous N-hydroxysuccinimide; and (c) subjecting the mixture after step (b) to tangential flow filtration, selective precipitation, non-adsorptive chromatography, adsorptive filtration, adsorptive chromatography, or a combination thereof, to purify the cell binding agent-cytotoxic agent conjugate from the other components of the mixture and thereby prepare a purified mixture of the cell binding agent-cytotoxic agent conjugate. The process optionally comprises comprising subjecting the mixture of step (a) to purification between steps (a)-(b) to purify the cell-binding agent-cytotoxic agent conjugates from the other components of the mixture and thereby prepare a purified mixture of the cell-binding agent-cytotoxic agent conjugates prior to the holding step (step (b)). The purification step between steps (a)-(b) optionally is carried out in the presence of exogenous NHS. Any purification method described herein can be used in the inventive process. In a preferred embodiment, tangential flow filtration, adsorptive chromatography, or non-adsorptive chromatography is utilized as the purification step.

In one embodiment, the cytotoxic agent-linker compound is prepared by contacting a cytotoxic agent with a bifunctional crosslinking reagent comprising a linker to covalently attach the cytotoxic agent to the linker. The cytotoxic agent-linker compound optionally is subjected to purification before contacting cytotoxic agent-linker compound with the cell-binding agent.

In one embodiment of the invention, the inventive process comprises two separate purification steps following the conjugation step. The purification steps following the conjugation reaction can be carried out in the presence of exogenous NHS. For example, the inventive process can comprise a conjugation step, followed by a purification step (in the absence or presence of exogenous NHS), followed by a holding step in the presence of exogenous NHS, followed by another purification step. Any purification method described herein can be used as the purification steps following the conjugation reaction. In a preferred embodiment, tangential flow filtration, adsorptive chromatography, non-adsorptive chromatography, or a combination thereof are utilized as the purification steps following the conjugation reaction.

Any suitable TFF systems may be utilized for purification, including a Pellicon type system (Millipore, Billerica, Mass.), a Sartocon Cassette system (Sartorius AG, Edgewood, N.Y.), and a Centrasette type system (Pall Corp., East Hills, N.Y.).

Any suitable adsorptive chromatography resin may be utilized for purification. Preferred adsorptive chromatography resins include hydroxyapatite chromatography, hydrophobic charge induction chromatography (HCIC), hydrophobic interaction chromatography (HIC), ion exchange chromatography, mixed mode ion exchange chromatography, immobilized metal affinity chromatography (IMAC), dye ligand chromatography, affinity chromatography, reversed phase chromatography, and combinations thereof. Examples of suitable hydroxyapatite resins include ceramic hydroxyapatite (CHT Type I and Type II, Bio-Rad Laboratories, Hercules, Calif.), HA Ultrogel hydroxyapatite (Pall Corp., East Hills, N.Y.), and ceramic fluoroapatite (CFT Type I and Type II, Bio-Rad Laboratories, Hercules, Calif.). An example of a suitable HCIC resin is MEP Hypercel resin (Pall Corp., East Hills, N.Y.). Examples of suitable HIC resins include Butyl-Sepharose, Hexyl-Sepaharose, Phenyl-Sepharose, and Octyl Sepharose resins (all from GE Healthcare, Piscataway, N.J.), as well as Macro-prep Methyl and Macro-Prep t-Butyl resins (Biorad Laboratories, Hercules, Calif.). Examples of suitable ion exchange resins include SP-Sepharose, CM-Sepharose, and Q-Sepharose resins (all from GE Healthcare, Piscataway, N.J.), and Unosphere S resin (Bio-Rad Laboratories, Hercules, Calif.). Examples of suitable mixed mode ion exchangers include Bakerbond ABx resin (JT Baker, Phillipsburg N.J.). Examples of suitable IMAC resins include Chelating Sepharose resin (GE Healthcare, Piscataway, N.J.) and Profinity IMAC resin (Bio-Rad Laboratories, Hercules, Calif.). Examples of suitable dye ligand resins include Blue Sepharose resin (GE Healthcare, Piscataway, N.J.) and Affi-gel Blue resin (Bio-Rad Laboratories, Hercules, Calif.). Examples of suitable affinity resins include Protein A Sepharose resin (e.g., MabSelect, GE Healthcare, Piscataway, N.J.), where the cell-binding agent is an antibody, and lectin affinity resins, e.g. Lentil Lectin Sepharose resin (GE Healthcare, Piscataway, N.J.), where the cell-binding agent bears appropriate lectin binding sites. Alternatively an antibody specific to the cell-binding agent may be used. Such an antibody can be immobilized to, for instance, Sepharose 4 Fast Flow resin (GE Healthcare, Piscataway, N.J.). Examples of suitable reversed phase resins include C4, C8, and C18 resins (Grace Vydac, Hesperia, CA).

Any suitable non-adsorptive chromatography resin may be utilized for purification. Examples of suitable non-adsorptive chromatography resins include, but are not limited to, SEPHADEX™ G-25, G-50, G-100, SEPHACRYL™ resins (e.g., S-200 and S-300), SUPERDEX™ resins (e.g., SUPERDEX™ 75 and SUPERDEX™ 200), BIO-GEL® resins (e.g., P-6, P-10, P-30, P-60, and P-100), and others known to those of ordinary skill in the art.

In one embodiment, the inventive process further comprises one or more (e.g., one, two, or three) holding steps in the presence of exogenous NHS (i.e., exogenous NHS is added to the holding step) to release the unstably bound linkers from the cell-binding agent. The holding step comprises holding the mixture after modification of the cell-binding agent with a bifunctional crosslinking reagent, after conjugation of a cytotoxic agent to the cell-binding agents having linkers bound thereto, and/or after a purification step.

The holding step comprises maintaining the solution at a suitable temperature (e.g., about 2° C. to about 37° C.) for a suitable period of time (e.g., about 1 hour to about 1 week) to release the unstably bound linkers from the cell-binding agent while not substantially releasing the stably bound linkers from the cell-binding agent. In one embodiment, the holding step comprises maintaining the solution at a low temperature (e.g., about 2° C. to about 10° C. or about 4° C.), at room temperature (e.g., about 20° C. to about 30° C. or about 20° C. to about 25° C.), or at an elevated temperature (e.g., about 30° C. to about 37° C.).

The duration of the holding step depends on the temperature at which the holding step is performed. For example, the duration of the holding step can be substantially reduced by performing the holding step at elevated temperature, with the maximum temperature limited by the stability of the cell-binding agent-cytotoxic agent conjugate. The holding step can comprise maintaining the solution for about 1 hour to about 1 day (e.g., about 1 hour, about 2 hours, about 3 hours, about 4 hours, about 5 hours, about 6 hours, about 7 hours, about 8 hours, about 9 hours, about 10 hours, about 12 hours, about 14 hours, about 16 hours, about 18 hours, about 20 hours, about 22 hours, or about 24 hours), about 5 hours to about 1 week, about 12 hours to about 1 week (e.g., about 12 hours, about 16 hours, about 20 hours, about 24 hours, about 2 days, about 3 days, about 4 days, about 5 days, about 6 days, or about 7 days), for about 12 hours to about 1 week (e.g., about 12 hours, about 16 hours, about 20 hours, about 24 hours, about 2 days, about 3 days, about 4 days, about 5 days, about 6 days, or about 7 days), or about 1 day to about 1 week.

In one embodiment, the holding step comprises maintaining the solution at a temperature of about 2° C. to about 8° C. for a period of at least about 12 hours for up to 1 day.

The pH value for the holding step preferably is about 4 to about 9 (e.g., about 4.5 to about 8.5 or about 5 to about 8). In one embodiment, the pH values for the holding step range from about 5 to about 7.5 (e.g., about 5.5 to about 7.5, about 6 to about 7.5, about 6.5 to about 7.5, about 7 to about 7.5, about 5 to about 7, about 5 to about 6.5, about 5 to about 5.5, about 5.5 to about 7, about 6 to about 6.5, or about 6 to about 7). For example, pH values for the holding step can be about 4, about 4.5, about 5, about 5.5, about 6, about 6.5, about 7, about 7.5, about 8, about 8.5, or about 9.

The holding step can be performed before or after the cell-binding agent is conjugated to the cytotoxic agent. In one embodiment, the holding step is performed directly after the modification of the cell-binding agent with the bifunctional crosslinking reagent. For example, the inventive process comprises a holding step after modification of the cell-binding agent with a bifunctional crosslinking reagent and before conjugation. After modification of the cell-binding agent, a purification step may be performed before the hold step and/or after the hold step, but prior to the conjugation step. In another embodiment, the holding step is performed directly after conjugation of the cytotoxic agent to the cell-binding agent having linkers bound thereto and prior to purification step. In another embodiment, the holding step is performed after the conjugation and purification steps and followed by an additional purification step.

In specific embodiments, the holding step can comprise incubating the mixture at a pH of about 5-7.5 or about 6.5-7.5 for about 1 hour to about 1 week at about 2° C. to about room temperature.

The invention provides a process for preparing compositions of stable conjugates comprising a cell-binding agent chemically coupled to a cytotoxic agent, wherein the compositions are substantially free of unstable conjugates. In this respect, the invention provides a process for preparing cell-binding agent-cytotoxic agent conjugate of substantially high purity and stability. Such compositions can be used for treating diseases because of the high purity and stability of the conjugates. Compositions comprising a cell-binding agent, such as an antibody, chemically coupled to a cytotoxic agent, such as a maytansinoid, are described in, for example, U.S. Pat. No. 7,374,762 and U.S. Patent Application Publication No. 2007/0031402. In one aspect of the invention, a cell-binding agent-cytotoxic agent conjugate of substantially high purity has one or more of the following features: (a) greater than about 90% (e.g., greater than or equal to about 91%, 92%, 93%, 94%, 95%, 96%, 97%, 98%, 99%, or 100%), preferably greater than about 95%, of conjugate species are monomeric, (b) unconjugated linker level in the conjugate preparation is less than about 10% (e.g., less than or equal to about 9%, 8%, 7%, 6%, 5%, 4%, 3%, 2%, 1%, or 0%) (relative to total linker), (c) less than 10% of conjugate species are crosslinked (e.g., less than or equal to about 9%, 8%, 7%, 6%, 5%, 4%, 3%, 2%, 1%, or 0%), (d) free cytotoxic agent level in the conjugate preparation is less than about 2% (e.g., less than or equal to about 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, 0.1%, or 0%) (relative to total cytotoxic agent), and/or (e) no substantial increase in free cytotoxic agent level upon storage (e.g., after about 1 week, about 2 weeks, about 3 weeks, about 1 month, about 2 months, about 3 months, about 4 months, about 5 months, about 6 months, about 1 year, about 2 years, or about 5 years). “Substantial increase” in free cytotoxic agent level means that after certain storage time, the increase in the level of free cytotoxic agent is less than about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.2%, about 2.5%, about 2.7%, about 3.0%, about 3.2%, about 3.5%, about 3.7%, or about 4.0%.

As used herein, the term “unconjugated linker” refers to the cell-binding agent that is covalently linked with the bifunctional crosslinking reagent, wherein the cell-binding agent is not covalently coupled to the cytotoxic agent through the linker of the bifunctional crosslinking reagent (i.e., the “unconjugated linker” can be represented by CBA-L, wherein CBA represents the cell-binding agent and L represents the bifunctional crosslinking reagent. In contrast, the cell-binding agent cytotoxic agent conjugate can be represented by CBA-L-D, wherein D represents the cytotoxic agent).

In one embodiment, the average molar ratio of the cytotoxic agent to the cell-binding agent in the cell-binding agent cytotoxic agent conjugate is about 1 to about 10, about 2 to about 7, about 3 to about 5, about 2.5 to about 4.5 (e.g., about 2.5, about 2.6, about 2.7, about 2.8, about 2.9, about 3.0, about 3.1, about 3.3, about 3.4, about 3.5, about 3.6, about 3.7, about 3.8, about 3.9, about 4.0, about 4.1, about 4.2, about 4.3, about 4.4, about 4.5), about 3.0 to about 4.0, about 3.2 to about 4.2, about 4.5 to 5.5 (e.g., about 4.5, about 4.6, about 4.7, about 4.8, about 4.9, about 5.0, about 5.1, about 5.2, about 5.3, about 5.4, about 5.5).

The cell-binding agent can be any suitable agent that binds to a cell, typically and preferably an animal cell (e.g., a human cell). The cell-binding agent preferably is a peptide or a polypeptide. Suitable cell-binding agents include, for example, antibodies (e.g., monoclonal antibodies and fragments thereof), interferons (e.g., alpha, beta, gamma), lymphokines (e.g., interleukin 2 (IL-2), interleukin 3 (IL-3), interleukin 4 (IL-4), interleukin 6 (IL-6), hormones (e.g., insulin, TRH (thyrotropin releasing hormone), MSH (melanocyte-stimulating hormone), steroid hormones, such as androgens and estrogens), growth factors and colony-stimulating factors, such as epidermal growth factor (EGF), transforming growth factor alpha (TGF-alpha), fibroblast growth factor (FGF), vascular endothelial growth factor (VEGF), colony stimulating factors (CSFs), such as G-CSF, M-CSF and GM-CSF (Burgess, Immunology Today 5:155-158 (1984)), nutrient-transport molecules (e.g., transferrin), vitamins (e.g., folate) and any other agent or molecule that specifically binds a target molecule on the surface of a cell.

Where the cell-binding agent is an antibody, it binds to an antigen that is a polypeptide and may be a transmembrane molecule (e.g. receptor) or a ligand such as a growth factor. Exemplary antigens include molecules such as renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor vmc, factor IX, tissue factor (TF), and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin, such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; protein A or D; rheumatoid factors; a neurotrophic factor such as bone-derived neurotrophic factor (BDNF), neurotrophin-3, -4, -5, or -6 (NT-3, NT4, NT-5, or NT-6), or a nerve growth factor such as NGF-β; platelet-derived growth factor (PDGF); fibroblast growth factor such as aFGF and bFGF; epidermal growth factor (EGF); transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins, EpCAM, GD3, FLT3, PSMA, PSCA, MUC1, MUC16, STEAP, CEA, TENB2, EphA receptors, EphB receptors, folate receptor, FOLR1, mesothelin, cripto, alphaᵥbeta₆, integrins, VEGF, VEGFR, EGFR, tarnsferrin receptor, IRTA1, IRTA2, IRTA3, IRTA4, IRTA5; CD proteins such as CD2, CD3, CD4, CD5, CD6, CD8, CD11, CD14, CD19, CD20, CD21, CD22, CD25, CD26, CD28, CD30, CD33, CD36, CD37, CD38, CD40, CD44, CD52, CD55, CD56, CD59, CD70, CD79, CD80. CD81, CD103, CD105, CD134, CD137, CD138, CD152, or an antibody which binds to one or more tumor-associated antigens or cell-surface receptors disclosed in U.S. Patent Application Publication No. 2008/0171040 or U.S. Patent Application Publication No. 2008/0305044, which are incorporated herein in their entirety by reference; erythropoietin; osteoinductive factors; immunotoxins; a bone morphogenetic protein (BMP); an interferon, such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the HIV envelope; transport proteins; homing receptors; addressins; regulatory proteins; integrins, such as CD11a, CD11b, CD11c, CD18, an ICAM, VLA-4 and VCAM; a tumor associated antigen such as HER2, HER3 or HER4 receptor; endoglin, c-Met, 1GF1R, PSGR, NGEP, PSMA, PSCA, LGR5, B7H4, and fragments of any of the above-listed polypeptides.

Additionally, GM-CSF, which binds to myeloid cells can be used as a cell-binding agent to diseased cells from acute myelogenous leukemia. IL-2 which binds to activated T-cells can be used for prevention of transplant graft rejection, for therapy and prevention of graft-versus-host disease, and for treatment of acute T-cell leukemia. MSH, which binds to melanocytes, can be used for the treatment of melanoma, as can antibodies directed towards melanomas. Folic acid can be used to target the folate receptor expressed on ovarian and other tumors. Epidermal growth factor can be used to target squamous cancers such as lung and head and neck. Somatostatin can be used to target neuroblastomas and other tumor types.

Cancers of the breast and testes can be successfully targeted with estrogen (or estrogen analogues) or androgen (or androgen analogues) respectively as cell-binding agents.

The term “antibody,” as used herein, refers to any immunoglobulin, any immunoglobulin fragment, such as Fab, Fab′, F(ab)₂, dsFv, sFv, minibodies, diabodies, tribodies, tetrabodies, nanobodies, probodies, domain bodies, unibodies and the like (Parham, J. Immunol. 131: 2895-2902 (1983); Spring et al., J. Immunol. 113: 470-478 (1974); Nisonoff et al., Arch. Biochem. Biophys. 89: 230-244 (1960); Kim et al., Mol. Cancer Ther., 7: 2486-2497 (2008); Carter, Nature Revs., 6: 343-357 (2006)), or immunoglobulin chimera, which can bind to an antigen on the surface of a cell (e.g., which contains a complementarity determining region (CDR)). Any suitable antibody can be used as the cell-binding agent. One of ordinary skill in the art will appreciate that the selection of an appropriate antibody will depend upon the cell population to be targeted. In this regard, the type and number of cell surface molecules (i.e., antigens) that are selectively expressed in a particular cell population (typically and preferably a diseased cell population) will govern the selection of an appropriate antibody for use in the inventive composition. Cell surface expression profiles are known for a wide variety of cell types, including tumor cell types, or, if unknown, can be determined using routine molecular biology and histochemistry techniques. The antibody can be bispecific antibodies (Morrison, S L, Nature biotechnology, 25(11): 1233-4 (2007)).

The antibody can be polyclonal or monoclonal, but is most preferably a monoclonal antibody. As used herein, “polyclonal” antibodies refer to heterogeneous populations of antibody molecules, typically contained in the sera of immunized animals. “Monoclonal” antibodies refer to homogenous populations of antibody molecules that are specific to a particular antigen. Monoclonal antibodies are typically produced by a single clone of B lymphocytes (“B cells”). Monoclonal antibodies may be obtained using a variety of techniques known to those skilled in the art, including standard hybridoma technology (see, e.g., Köhler and Milstein, Eur. J. Immunol., 5: 511-519 (1976); Harlow and Lane (eds.), Antibodies: A Laboratory Manual, CSH Press (1988); and C. A. Janeway et al. (eds.), Immunobiology, 5^{th} Ed., Garland Publishing, New York, N.Y. (2001)). In brief, the hybridoma method of producing monoclonal antibodies typically involves injecting any suitable animal, typically and preferably a mouse, with an antigen (i.e., an “immunogen”). The animal is subsequently sacrificed, and B cells isolated from its spleen are fused with human myeloma cells. A hybrid cell is produced (i.e., a “hybridoma”), which proliferates indefinitely and continuously secretes high titers of an antibody with the desired specificity in vitro. Any appropriate method known in the art can be used to identify hybridoma cells that produce an antibody with the desired specificity. Such methods include, for example, enzyme-linked immunosorbent assay (ELISA), Western blot analysis, and radioimmunoassay. The population of hybridomas is screened to isolate individual clones, each of which secretes a single antibody species to the antigen. Because each hybridoma is a clone derived from fusion with a single B cell, all the antibody molecules it produces are identical in structure, including their antigen binding site and isotype. Monoclonal antibodies also may be generated using other suitable techniques including EBV-hybridoma technology (see, e.g., Haskard and Archer, J. Immunol. Methods, 74(2): 361-67 (1984), and Roder et al., Methods Enzymol., 121: 140-67 (1986)), bacteriophage vector expression systems (see, e.g., Huse et al., Science, 246: 1275-81 (1989)), or phage display libraries comprising antibody fragments, such as Fab and scFv (single chain variable region) (see, e.g., U.S. Pat. Nos. 5,885,793 and 5,969,108, and International Patent Application Publication Nos. WO 92/01047 and WO 99/06587).

The monoclonal antibody can be isolated from or produced in any suitable animal, but is preferably produced in a mammal, more preferably a mouse or human, and most preferably a human. Methods for producing an antibody in mice are well known to those skilled in the art and are described herein. With respect to human antibodies, one of ordinary skill in the art will appreciate that polyclonal antibodies can be isolated from the sera of human subjects vaccinated or immunized with an appropriate antigen. Alternatively, human antibodies can be generated by adapting known techniques for producing human antibodies in non-human animals such as mice (see, e.g., U.S. Pat. Nos. 5,545,806; 5,569,825; and 5,714,352, and U.S. Patent Application Publication No. 2002/0197266 A1).

While being the ideal choice for therapeutic applications in humans, human antibodies, particularly human monoclonal antibodies, typically are more difficult to generate than mouse monoclonal antibodies. Mouse monoclonal antibodies, however, induce a rapid host antibody response when administered to humans, which can reduce the therapeutic or diagnostic potential of the antibody-cytotoxic agent conjugate. To circumvent these complications, a monoclonal antibody preferably is not recognized as “foreign” by the human immune system.

To this end, phage display can be used to generate the antibody. In this regard, phage libraries encoding antigen-binding variable (V) domains of antibodies can be generated using standard molecular biology and recombinant DNA techniques (see, e.g., Sambrook et al. (eds.), Molecular Cloning, A Laboratory Manual, 3^{rd} Edition, Cold Spring Harbor Laboratory Press, New York (2001)). Phage encoding a variable region with the desired specificity are selected for specific binding to the desired antigen, and a complete human antibody is reconstituted comprising the selected variable domain. Nucleic acid sequences encoding the reconstituted antibody are introduced into a suitable cell line, such as a myeloma cell used for hybridoma production, such that human antibodies having the characteristics of monoclonal antibodies are secreted by the cell (see, e.g., Janeway et al., supra, Huse et al., supra, and U.S. Pat. No. 6,265,150). Alternatively, monoclonal antibodies can be generated from mice that are transgenic for specific human heavy and light chain immunoglobulin genes. Such methods are known in the art and described in, for example, U.S. Pat. Nos. 5,545,806 and 5,569,825, and Janeway et al., supra.

Most preferably the antibody is a humanized antibody. As used herein, a “humanized” antibody is one in which the complementarity-determining regions (CDR) of a mouse monoclonal antibody, which form the antigen binding loops of the antibody, are grafted onto the framework of a human antibody molecule. Owing to the similarity of the frameworks of mouse and human antibodies, it is generally accepted in the art that this approach produces a monoclonal antibody that is antigenically identical to a human antibody but binds the same antigen as the mouse monoclonal antibody from which the CDR sequences were derived. Methods for generating humanized antibodies are well known in the art and are described in detail in, for example, Janeway et al., supra, U.S. Pat. Nos. 5,225,539, 5,585,089 and 5,693,761, European Patent 0239400 B1, and United Kingdom Patent 2188638. Humanized antibodies can also be generated using the antibody resurfacing technology described in U.S. Pat. No. 5,639,641 and Pedersen et al., J. Mol. Biol., 235: 959-973 (1994). While the antibody employed in the conjugate of the inventive composition most preferably is a humanized monoclonal antibody, a human monoclonal antibody and a mouse monoclonal antibody, as described above, are also within the scope of the invention.

Antibody fragments that have at least one antigen binding site, and thus recognize and bind to at least one antigen or receptor present on the surface of a target cell, also are within the scope of the invention. In this respect, proteolytic cleavage of an intact antibody molecule can produce a variety of antibody fragments that retain the ability to recognize and bind antigens. For example, limited digestion of an antibody molecule with the protease papain typically produces three fragments, two of which are identical and are referred to as the Fab fragments, as they retain the antigen binding activity of the parent antibody molecule. Cleavage of an antibody molecule with the enzyme pepsin normally produces two antibody fragments, one of which retains both antigen-binding arms of the antibody molecule, and is thus referred to as the F(ab′)₂ fragment. Reduction of a F(ab′)₂ fragment with dithiothreitol or mercaptoethylamine produces a fragment referred to as a Fab′ fragment. A single-chain variable region fragment (sFv) antibody fragment, which consists of a truncated Fab fragment comprising the variable (V) domain of an antibody heavy chain linked to a V domain of a light antibody chain via a synthetic peptide, can be generated using routine recombinant DNA technology techniques (see, e.g., Janeway et al., supra). Similarly, disulfide-stabilized variable region fragments (dsFv) can be prepared by recombinant DNA technology (see, e.g., Reiter et al., Protein Engineering, 7: 697-704 (1994)). Antibody fragments in the context of the invention, however, are not limited to these exemplary types of antibody fragments. Any suitable antibody fragment that recognizes and binds to a desired cell surface receptor or antigen can be employed. Antibody fragments are further described in, for example, Parham, J. Immunol., 131: 2895-2902 (1983); Spring et al., J. Immunol., 113: 470-478 (1974); and Nisonoff et al., Arch. Biochem. Biophys., 89: 230-244 (1960). Antibody-antigen binding can be assayed using any suitable method known in the art, such as, for example, radioimmunoassay (RIA), ELISA, Western blot, immunoprecipitation, and competitive inhibition assays (see, e.g., Janeway et al., supra, and U.S. Patent Application Publication No. 2002/0197266 A1).

In addition, the antibody can be a chimeric antibody or an antigen binding fragment thereof. By “chimeric” it is meant that the antibody comprises at least two immunoglobulins, or fragments thereof, obtained or derived from at least two different species (e.g., two different immunoglobulins, such as a human immunoglobulin constant region combined with a murine immunoglobulin variable region). The antibody also can be a domain antibody (dAb) or an antigen binding fragment thereof, such as, for example, a camelid antibody (see, e.g., Desmyter et al., Nature Struct. Biol., 3: 752, (1996)), or a shark antibody, such as, for example, a new antigen receptor (IgNAR) (see, e.g., Greenberg et al., Nature, 374: 168 (1995), and Stanfield et al., Science, 305: 1770-1773 (2004)).

Any suitable antibody can be used in the context of the invention. For example, the monoclonal antibody J5 is a murine IgG2a antibody that is specific for Common Acute Lymphoblastic Leukemia Antigen (CALLA) (Ritz et al., Nature, 283: 583-585 (1980)), and can be used to target cells that express CALLA (e.g., acute lymphoblastic leukemia cells). The monoclonal antibody MY9 is a murine IgG1 antibody that binds specifically to the CD33 antigen (Griffin et al., Leukemia Res., 8: 521 (1984)), and can be used to target cells that express CD33 (e.g., acute myelogenous leukemia (AML) cells).

Similarly, the monoclonal antibody anti-B4 (also referred to as B4) is a murine IgG1 antibody that binds to the CD19 antigen on B cells (Nadler et al., J. Immunol., 131: 244-250 (1983)), and can be used to target B cells or diseased cells that express CD19 (e.g., non-Hodgkin's lymphoma cells and chronic lymphoblastic leukemia cells). N901 is a murine monoclonal antibody that binds to the CD56 (neural cell adhesion molecule) antigen found on cells of neuroendocrine origin, including small cell lung tumor, which can be used in the conjugate to target drugs to cells of neuroendocrine origin. The J5, MY9, and B4 antibodies preferably are resurfaced or humanized prior to their use as part of the conjugate. Resurfacing or humanization of antibodies is described in, for example, Roguska et al., Proc. Natl. Acad. Sci. USA, 91: 969-73 (1994).

In addition, the monoclonal antibody C242 binds to the CanAg antigen (see, e.g., U.S. Pat. No. 5,552,293), and can be used to target the conjugate to CanAg expressing tumors, such as colorectal, pancreatic, non-small cell lung, and gastric cancers. HuC242 is a humanized form of the monoclonal antibody C242 (see, e.g., U.S. Pat. No. 5,552,293). The hybridoma from which HuC242 is produced is deposited with ECACC identification Number 90012601. HuC242 can be prepared using CDR-grafting methodology (see, e.g., U.S. Pat. Nos. 5,585,089, 5,693,761, and 5,693,762) or resurfacing technology (see, e.g., U.S. Pat. No. 5,639,641). HuC242 can be used to target the conjugate to tumor cells expressing the CanAg antigen, such as, for example, colorectal, pancreatic, non-small cell lung, and gastric cancer cells.

To target ovarian cancer and prostate cancer cells, an anti-MUC1 antibody can be used as the cell-binding agent in the conjugate. Anti-MUC1 antibodies include, for example, anti-HMFG-2 (see, e.g., Taylor-Papadimitriou et al., Int. J. Cancer, 28: 17-21 (1981)), hCTM01 (see, e.g., van Hof et al., Cancer Res., 56: 5179-5185 (1996)), and DS6. Prostate cancer cells also can be targeted with the conjugate by using an anti-prostate-specific membrane antigen (PSMA) as the cell-binding agent, such as J591 (see, e.g., Liu et al., Cancer Res., 57: 3629-3634 (1997)). Moreover, cancer cells that express the Her2 antigen, such as breast, prostate, and ovarian cancers, can be targeted using the antibody trastuzumab. Anti-IGF-IR antibodies that bind to insulin-like growth factor receptor also can be used in the conjugate. Antibodies that bind to CD27L, EGFRvIII, Cripto, CD138, CD38, EphA2, integrins, CD37, folate receptor, and Her3 also can be used in the conjugate.

In one embodiment, the antibody is selected from the group consisting of huN901, huMy9-6, huB4, huC242, trastuzumab, bivatuzumab, sibrotuzumab, rituximab, huDS6, anti-mesothelin antibodies described in International Patent Application Publication No. WO 2010/124797 (such as MF-T), anti-cripto antibodies described in U.S. Patent Application Publication No. 2010/0093980 (such as huB3F6), anti-CD138 antibodies described in U.S. Patent Application Publication No. 2007/0183971 (such as huB-B4), anti-EGFRvIII antibodies described U.S. Pat. Nos. 7,736,644 and 7,628,986 and U.S. Patent Application Publication Nos. 2010/0111979; 2009/0240038; 2009/0175887; 2009/0156790; and 2009/0155282, humanized EphA2 antibodies described in International Patent Application Publication Nos. WO/2011/039721 and WO/2011/039724 (such as 2H11R35R74); anti-CD38 antibodies described in International Patent Application Publication No. WO 2008/047242 (such as hu38SB19), anti-folate receptor antibodies described in U.S. Provisional Application Nos. 61/307,797, 61/346,595 and 61/413,172, and U.S. Patent Application Publication No. 2012/0009181 (e.g., huMov19); anti-IGF1R antibodies described in U.S. Pat. Nos. 5,958,872 and 6,596,743; anti-CD37 antibodies described in U.S. Patent Application Publication No. 2011/0256153 (e.g., huCD37-3); anti-integrin αᵥβ₆antibodies described in U.S. Patent Application Publication No. 2006/0127407 (e.g., CNTO95); and anti-Her3 antibodies described in International Patent Application Publication No. WO 2012/019024. Particularly preferred antibodies are humanized monoclonal antibodies described herein.

While the cell-binding agent preferably is an antibody, the cell-binding agent also can be a non-antibody molecule. Suitable non-antibody molecules include, for example, ankyrin repeat proteins (DARPins; Zahnd et al., J. Biol. Chem., 281, 46, 35167-35175, (2006); Binz et al., Nature Biotechnology, 23: 1257-1268 (2005)) or ankyrin-like repeats proteins or synthetic peptides described, for example, in U.S. Patent Application Publication No. 2007/0238667; U.S. Pat. No. 7,101,675; and International Patent Application Publication Nos. WO/2007/147213 and WO/2007/062466), interferons (e.g., alpha, beta, or gamma interferon), lymphokines (e.g., interleukin 2 (IL-2), IL-3, IL-4, or IL-6), hormones (e.g., insulin), growth factors (e.g., EGF, TGF-alpha, FGF, and VEGF), colony-stimulating factors (e.g., G-CSF, M-CSF, and GM-CSF (see, e.g., Burgess, Immunology Today, 5: 155-158 (1984)), somatostatin, and transferrin (see, e.g., O'Keefe et al., J. Biol. Chem., 260: 932-937 (1985)). For example, GM-CSF, which binds to myeloid cells, can be used as a cell-binding agent to target acute myelogenous leukemia cells. In addition, IL-2, which binds to activated T-cells, can be used for prevention of transplant graft rejection, for therapy and prevention of graft-versus-host disease, and for treatment of acute T-cell leukemia. Epidermal growth factor (EGF) can be used to target squamous cancers such as lung cancer and head and neck cancer. Somatostatin can be used to target neuroblastoma cells and other tumor cell types.

The conjugate can comprise any suitable cytotoxic agent. A “cytotoxic agent,” as used herein, refers to any compound that results in the death of a cell, induces cell death, or decreases cell viability. Suitable cytotoxic agents include, for example, maytansinoids and maytansinoid analogs, taxoids, CC-1065 and CC-1065 analogs, and dolastatin and dolastatin analogs. In a preferred embodiment of the invention, the cytotoxic agent is a maytansinoid, including maytansinol and maytansinol analogs. Maytansinoids are compounds that inhibit microtubule formation and are highly toxic to mammalian cells. Examples of suitable maytansinol analogues include those having a modified aromatic ring and those having modifications at other positions. Such maytansinoids are described in, for example, U.S. Pat. Nos. 4,256,746; 4,294,757; 4,307,016; 4,313,946; 4,315,929; 4,322,348; 4,331,598; 4,361,650; 4,362,663; 4,364,866; 4,424,219; 4,371,533; 4,450,254; 5,475,092; 5,585,499; 5,846,545; and 6,333,410.

Examples of maytansinol analogs having a modified aromatic ring include: (1) C-19-dechloro (U.S. Pat. No. 4,256,746) (prepared by LAH reduction of ansamytocin P2), (2) C-20-hydroxy (or C-20-demethyl) +/−C-19-dechloro (U.S. Pat. Nos. 4,361,650 and 4,307,016) (prepared by demethylation using Streptomycesor Actinomycesor dechlorination using LAH), and (3) C-20-demethoxy, C-20-acyloxy (—OCOR), +/−dechloro (U.S. Pat. No. 4,294,757) (prepared by acylation using acyl chlorides).

Examples of maytansinol analogs having modifications of positions other than an aromatic ring include: (1) C-9-SH (U.S. Pat. No. 4,424,219) (prepared by the reaction of maytansinol with H₂S or P₂S₅), (2) C-14-alkoxymethyl (demethoxy/CH₂OR) (U.S. Pat. No. 4,331,598), (3) C-14-hydroxymethyl or acyloxymethyl (CH₂OH or CH₂OAc) (U.S. Pat. No. 4,450,254) (prepared from Nocardia), (4) C-15-hydroxy/acyloxy (U.S. Pat. No. 4,364,866) (prepared by the conversion of maytansinol by Streptomyces), (5) C-15-methoxy (U.S. Pat. Nos. 4,313,946 and 4,315,929) (isolated from Trewia nudiflora), (6) C-18-N-demethyl (U.S. Pat. Nos. 4,362,663 and 4,322,348) (prepared by the demethylation of maytansinol by Streptomyces), and (7) 4,5-deoxy (U.S. Pat. No. 4,371,533) (prepared by the titanium trichloride/LAH reduction of maytansinol).

In a preferred embodiment of the invention, the conjugate utilizes the thiol-containing maytansinoid DM1, also known as N^{2′}-deacetyl-N^{2′}-(3-mercapto-1-oxopropyl)-maytansine, as the cytotoxic agent. The structure of DM1 is represented by formula (I):

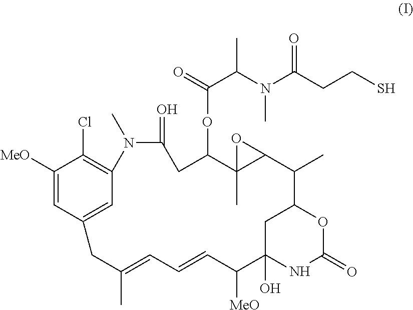 [SMILES: COc1cc2cc(c1Cl)N(C)C(=O)CC(OC(=O)C(C)N(C)C(=O)CCS)C1(C)OC1C(C)C1CC(O)(NC(=O)O1)C(OC)/C=C/C=C(\C)C2]

In another preferred embodiment of the invention, the conjugate utilizes the thiol-containing maytansinoid DM4, also known as N^{2′}-deacetyl-N^{2′}-(4-methyl-4-mercapto-1-oxopentyl)-maytansine, as the cytotoxic agent. The structure of DM4 is represented by formula (II):

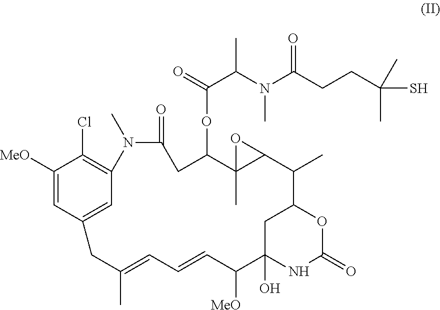 [SMILES: COc1cc2cc(c1Cl)N(C)C(=O)CC(OC(=O)C(C)N(C)C(=O)CCC(C)(C)S)C1(C)OC1C(C)C1CC(O)(NC(=O)O1)C(OC)/C=C/C=C(\C)C2]

Other maytansines may be used in the context of the invention, including, for example, thiol and disulfide-containing maytansinoids bearing a mono or di-alkyl substitution on the carbon atom bearing the sulfur atom. Particularly preferred is a maytansinoid having at the C-3 position (a)C-14 hydroxymethyl, C-15 hydroxy, or C-20 desmethyl functionality, and (b) an acylated amino acid side chain with an acyl group bearing a hindered sulfhydryl group, wherein the carbon atom of the acyl group bearing the thiol functionality has one or two substituents, said substituents being CH₃, C₂H₅, linear or branched alkyl or alkenyl having from 1 to 10 carbon atoms, cyclic alkyl or alkenyl having from 3 to 10 carbon atoms, phenyl, substituted phenyl, or heterocyclic aromatic or heterocycloalkyl radical, and further wherein one of the substituents can be H, and wherein the acyl group has a linear chain length of at least three carbon atoms between the carbonyl functionality and the sulfur atom.

Additional maytansinoids for use in the context of the invention include compounds represented by formula (III):

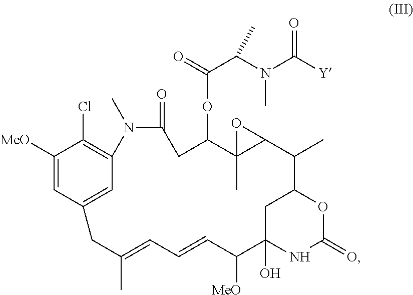

wherein Y′ represents
(CR₇R₈)ₗ(CR₉═CR₁₀)ₚ(C≡C)_{q}Aₒ(CR₅R₆)ₘDᵤ(CR₁₁═CR₁₂)ᵣ(C≡C)ₛBₜ(CR₃R₄)ₙCR₁R₂SZ,
wherein R₁ and R₂ are each independently CH₃, C₂H₅, linear alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical, and wherein R₂ also can be H,
wherein A, B, D are cycloalkyl or cycloalkenyl having 3-10 carbon atoms, simple or substituted aryl, or heterocyclic aromatic, or heterocycloalkyl radical,
wherein R₃, R₄, R₅, R₆, R₇, R₈, R₉, R₁₀, R₁₁, and R₁₂ are each independently H, CH₃, C₂H₅, linear alkyl or alkenyl having from 1 to 10 carbon atoms, branched or cyclic alkyl or alkenyl having from 3 to 10 carbon atoms, phenyl, substituted phenyl or heterocyclic aromatic, or heterocycloalkyl radical,
wherein l, m, n, o, p, q, r, s, and t are each independently zero or an integer from 1 to 5, provided that at least two of l, m, n, o, p, q, r, s and t are not zero at any one time, and wherein Z is H, SR or COR, wherein R is linear alkyl or alkenyl having from 1 to 10 carbon atoms, branched or cyclic alkyl or alkenyl having from 3 to 10 carbon atoms, or simple or substituted aryl or heterocyclic aromatic, or heterocycloalkyl radical.

Preferred embodiments of formula (III) include compounds of formula (III) wherein (a) R₁ is H, R₂ is methyl and Z is H, (b) R₁ and R₂ are methyl and Z is H, (c) R₁ is H, R₂ is methyl, and Z is —SCH₃, and (d) R₁ and R₂ are methyl, and Z is —SCH₃.

Such additional maytansinoinds also include compounds represented by formula (IV-L), (IV-D), or (IV-D,L):

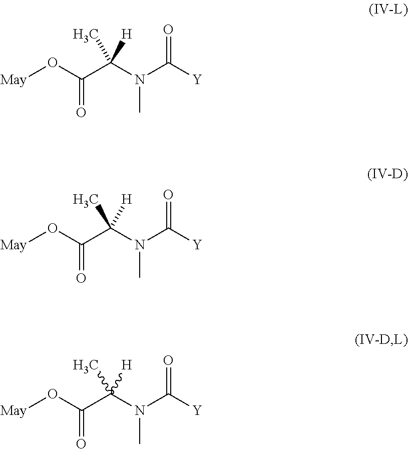

wherein Y represents (CR₇R₈)₁(CR₅R₆)ₘ(CR₃R₄)ₙCR₁R₂SZ,
wherein R₁ and R₂ are each independently CH₃, C₂H₅, linear alkyl, or alkenyl having from 1 to 10 carbon atoms, branched or cyclic alkyl or alkenyl having from 3 to 10 carbon atoms, phenyl, substituted phenyl, or heterocyclic aromatic or heterocycloalkyl radical, and wherein R₂ also can be H,
wherein R₃, R₄, R₅, R₆, R₇, and R₈ are each independently H, CH₃, C₂H₅, linear alkyl or alkenyl having from 1 to 10 carbon atoms, branched or cyclic alkyl or alkenyl having from 3 to 10 carbon atoms, phenyl, substituted phenyl, or heterocyclic aromatic or heterocycloalkyl radical,
wherein l, m, and n are each independently an integer of from 1 to 5, and in addition n can be zero,
wherein Z is H, SR, or COR wherein R is linear or branched alkyl or alkenyl having from 1 to 10 carbon atoms, cyclic alkyl or alkenyl having from 3 to 10 carbon atoms, or simple or substituted aryl or heterocyclic aromatic or heterocycloalkyl radical, and
wherein May represents a maytansinoid which bears the side chain at C-3, C-14 hydroxymethyl, C-15 hydroxy, or C-20 desmethyl.

Preferred embodiments of formulas (IV-L), (IV-D) and (IV-D,L) include compounds of formulas (IV-L), (IV-D) and (IV-D,L) wherein (a) R₁ is H, R₂ is methyl, R₅, R₆, R₇, and R₈ are each H, l and m are each 1, n is 0, and Z is H, (b) R₁ and R₂ are methyl, R₅, R₆, R₇, R₈ are each H, l and m are 1, n is 0, and Z is H, (c) R₁ is H, R₂ is methyl, R₅, R₆, R₇, and R₈ are each H, l and m are each 1, n is 0, and Z is —SCH₃, or (d) R₁ and R₂ are methyl, R₅, R₆, R₇, R₈ are each H, l and m are 1, n is 0, and Z is —SCH₃.

Preferably the cytotoxic agent is represented by formula (IV-L).

Additional preferred maytansinoids also include compounds represented by formula (V):

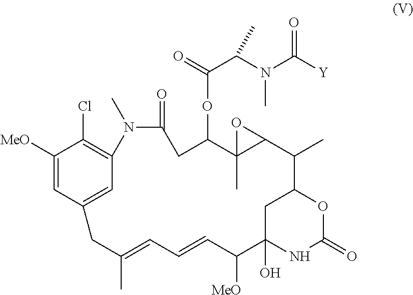

wherein Y represents (CR₇R₈)ₗ(CR₅R₆)ₘ(CR₃R₄)ₙCR₁R₂SZ,
wherein R₁ and R₂ are each independently CH₃, C₂H₅, linear alkyl, or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical, and wherein R₂ also can be H,
wherein R₃, R₄, R₅, R₆, R₇, and R₈ are each independently H, CH₃, C₂H₅, linear alkyl or alkenyl having from 1 to 10 carbon atoms, branched or cyclic alkyl or alkenyl having from 3 to 10 carbon atoms, phenyl, substituted phenyl, or heterocyclic aromatic or heterocycloalkyl radical,
wherein l, m, and n are each independently an integer of from 1 to 5, and in addition n can be zero, and
wherein Z is H, SR or COR, wherein R is linear alkyl or alkenyl having from 1 to 10 carbon atoms, branched or cyclic alkyl or alkenyl having from 3 to 10 carbon atoms, or simple or substituted aryl or heterocyclic aromatic or heterocycloalkyl radical.

Preferred embodiments of formula (V) include compounds of formula (V) wherein (a) R₁ is H, R₂ is methyl, R₅, R₆, R₇, and R₈ are each H; l and m are each 1; n is 0; and Z is H, (b) R₁ and R₂ are methyl; R₅, R₆, R₇, R₈ are each H, l and m are 1; n is 0; and Z is H, (c) R₁ is H, R₂ is methyl, R₅, R₆, R₇, and R₈ are each H, l and m are each 1, n is 0, and Z is —SCH₃, or (d) R₁ and R₂ are methyl, R₅, R₆, R₇, R₈ are each H, l and m are 1, n is 0, and Z is —SCH₃.

Still further preferred maytansinoids include compounds represented by formula (VI-L), (VI-D), or (VI-D,L):

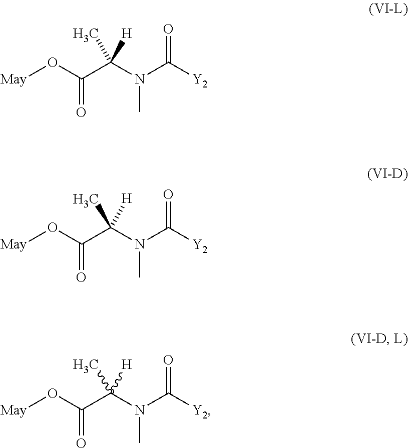

wherein Y₂ represents (CR₇R₈)ₗ(CR₅R₆)ₘ(CR₃R₄)ₙCR₁R₂SZ₂,
wherein R₁ and R₂ are each independently CH₃, C₂H₅, linear alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical, and wherein R₂ also can be H,
wherein R₃, R₄, R₅, R₆, R₇, and R₈ are each independently H, CH₃, C₂H₅, linear cyclic alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical,
wherein l, m, and n are each independently an integer of from 1 to 5, and in addition n can be zero,
wherein Z₂ is SR or COR, wherein R is linear alkyl or alkenyl having from 1 to 10 carbon atoms, branched or cyclic alkyl or alkenyl having from 3 to 10 carbon atoms, or simple or substituted aryl or heterocyclic aromatic or heterocycloalkyl radical, and wherein May is a maytansinoid.

In addition to maytansinoids, the cytotoxic agent used in the conjugate can be a taxane or derivative thereof. Taxanes are a family of compounds that includes paclitaxel (Taxol®), a cytotoxic natural product, and docetaxel (Taxotere®), a semi-synthetic derivative, which are both widely used in the treatment of cancer. Taxanes are mitotic spindle poisons that inhibit the depolymerization of tubulin, resulting in cell death. While docetaxel and paclitaxel are useful agents in the treatment of cancer, their antitumor activity is limited because of their non-specific toxicity towards normal cells. Further, compounds like paclitaxel and docetaxel themselves are not sufficiently potent to be used in conjugates of cell-binding agents.

A preferred taxane for use in the preparation of a cytotoxic conjugate is the taxane of formula (VIII):

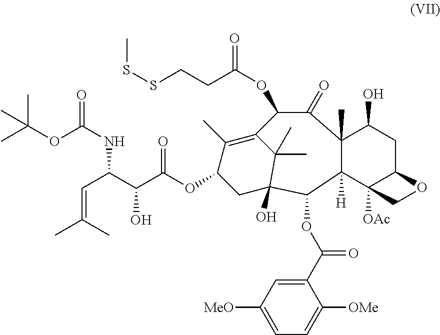 [SMILES: COc1ccc(OC)c(C(=O)O[C@H]2[C@@H]3[C@]4(OC(C)=O)CO[C@@H]4C[C@H](O)[C@@]3(C)C(=O)[C@H](OC(=O)CCSSC)C3=C(C)[C@@H](OC(=O)[C@H](O)[C@H](C=C(C)C)NC(=O)OC(C)(C)C)C[C@]2(O)C3(C)C)c1]

Methods for synthesizing taxanes that can be used in the context of the invention, along with methods for conjugating taxanes to cell-binding agents such as antibodies, are described in detail in U.S. Pat. Nos. 5,416,064; 5,475,092; 6,340,701; 6,372,738; 6,436,931; 6,596,757; 6,706,708; 6,716,821; and 7,390,898.

The cytotoxic also can be CC-1065 or a derivative thereof. CC-1065 is a potent anti-tumor antibiotic isolated from the culture broth of Streptomyces zelensis. CC-1065 is about 1000-fold more potent in vitro than commonly used anti-cancer drugs, such as doxorubicin, methotrexate, and vincristine (Bhuyan et al., Cancer Res., 42: 3532-3537 (1982)). CC-1065 and its analogs are disclosed in U.S. Pat. Nos. 5,585,499; 5,846,545; 6,340,701; and 6,372,738. The cytotoxic potency of CC-1065 has been correlated with its alkylating activity and its DNA-binding or DNA-intercalating activity. These two activities reside in separate parts of the molecule. In this respect, the alkylating activity is contained in the cyclopropapyrroloindole (CPI) subunit and the DNA-binding activity resides in the two pyrroloindole subunits of CC-1065.

Several CC-1065 analogs are known in the art and also can be used as the cytotoxic agent in the conjugate (see, e.g., Warpehoski et al., J. Med. Chem., 31: 590-603 (1988)). A series of CC-1065 analogs has been developed in which the CPI moiety is replaced by a cyclopropabenzindole (CBI) moiety (Boger et al., J. Org. Chem., 55: 5823-5833 (1990), and Boger et al., Bioorg. Med. Chem. Lett., 1: 115-120 (1991)). These CC-1065 analogs maintain the high in vitro potency of the parental drug, without causing delayed toxicity in mice. Like CC-1065, these compounds are alkylating agents that covalently bind to the minor groove of DNA to cause cell death.

The therapeutic efficacy of CC-1065 analogs can be greatly improved by changing the in vivo distribution through targeted delivery to a tumor site, resulting in lower toxicity to non-targeted tissues, and thus, lower systemic toxicity. To this end, conjugates of analogs and derivatives of CC-1065 with cell-binding agents that specifically target tumor cells have been generated (see, e.g., U.S. Pat. Nos. 5,475,092; 5,585,499; and 5,846,545). These conjugates typically display high target-specific cytotoxicity in vitro, and anti-tumor activity in human tumor xenograft models in mice (see, e.g., Chari et al., Cancer Res., 55: 4079-4084 (1995)).

Methods for synthesizing CC-1065 analogs are described in detail in U.S. Pat. Nos. 5,475,092; 5,585,499; 5,846,545; 6,534,660; 6,586,618; 6,756,397; and 7,329,760.

Drugs such as methotrexate, daunorubicin, doxorubicin, vincristine, vinblastine, melphalan, mitomycin C, chlorambucil, calicheamicin, tubulysin and tubulysin analogs, duocarmycin and duocarmycin analogs, dolastatin and dolastatin analogs also can be used as the cytotoxic agents of the invention. Doxarubicin and daunorubicin compounds (see, e.g., U.S. Pat. No. 6,630,579) can also be used as the cytotoxic agent.

The cell-binding agent cytotoxic agent conjugates may be prepared by in vitro methods. In order to link a cytotoxic agent to the antibody, a linking group is used. Suitable linking groups are well known in the art and include disulfide groups, acid labile groups, photolabile groups, peptidase labile groups, and esterase labile groups, as well as noncleavable linking groups.

In accordance with the invention, the cell-binding agent is modified by reacting a bifunctional crosslinking reagent with the cell-binding agent, thereby resulting in the covalent attachment of a linker molecule to the cell-binding agent. As used herein, a “bifunctional crosslinking reagent” refers to a reagent that possesses two reactive groups; one of which is capable of reacting with a cell-binding agent, while the other one is capable of reacting with the cytotoxic agent to link the cell-binding agent with the cytotoxic agent, thereby forming a conjugate.

Any suitable bifunctional crosslinking reagent can be used in connection with the invention, so long as the linker reagent provides for retention of the therapeutic, e.g., cytotoxicity, and targeting characteristics of the cytotoxic agent and the cell-binding agent, respectively, while providing an acceptable toxicity profile. Preferably, the linker molecule joins the cytotoxic agent to the cell-binding agent through chemical bonds (as described above), such that the cytotoxic agent and the cell-binding agent are chemically coupled (e.g., covalently bonded) to each other.

In one embodiment, the cell binding agent is chemically coupled to the cytotoxic agent via chemical bonds selected from the group consisting of disulfide bonds, acid labile bonds, photolabile bonds, peptidase labile bonds, and esterase labile bonds.

In one embodiment, the bifunctional crosslinking reagent comprises non-cleavable linkers. A non-cleavable linker is any chemical moiety that is capable of linking a cytotoxic agent, such as a maytansinoid, a taxane, or a CC-1065 analog, to a cell-binding agent in a stable, covalent manner. Thus, non-cleavable linkers are substantially resistant to acid-induced cleavage, light-induced cleavage, peptidase-induced cleavage, esterase-induced cleavage, and disulfide bond cleavage, at conditions under which the cytotoxic agent or the cell-binding agent remains active.

Suitable crosslinking reagents that form non-cleavable linkers between a cytotoxic agent and the cell-binding agent are well known in the art. In one embodiment, the cytotoxic agent is chemically coupled to the cell-binding agent through a thioether bond. Examples of non-cleavable linkers include linkers having a maleimido-based moeity or a haloacetyl-based moiety for reaction with the cytotoxic agent. Such bifunctional crosslinking agents are well known in the art (see U.S. Patent Application Publication Nos. 2010/0129314; 2009/0274713; 2008/0050310; 2005/0169933; and Pierce Biotechnology Inc. P.O. Box 117, Rockland, Ill. 61105, USA) and include, but not limited to, N-succinimidyl 4-(maleimidomethyl)cyclohexanecarboxylate (SMCC), N-succinimidyl-4-(N-maleimidomethyl)-cyclohexane-1-carboxy-(6-amidocaproate), which is a “long chain” analog of SMCC (LC-SMCC), κ-maleimidoundecanoic acid N-succinimidyl ester (KMUA), γ-maleimidobutyric acid N-succinimidyl ester (GMBS), ε-maleimidocaproic acid N-hydroxysuccinimide ester (EMCS), m-maleimidobenzoyl-N-hydroxysuccinimide ester (MBS), N-(α-maleimidoacetoxy)-succinimide ester (AMAS), succinimidyl-6-(β-maleimidopropionamido)hexanoate (SMPH), N-succinimidyl 4-(p-maleimidophenyl)-butyrate (SMPB), and N-(p-maleimidophenyl)isocyanate (PMPI). Cross-linking reagents comprising a haloacetyl-based moiety include N-succinimidyl-4-(iodoacetyl)-aminobenzoate (SIAB), N-succinimidyl iodoacetate (SIA), N-succinimidyl bromoacetate (SBA), and N-succinimidyl 3-(bromoacetamido)propionate (SBAP), bis-maleimidopolyethyleneglycol (BMPEO), BM(PEO)₂, BM(PEO)₃, N-(β-maleimidopropyloxy)succinimide ester (BMPS), 5-maleimidovaleric acid NHS, HBVS, 4-(4-N-maleimidophenyl)-butyric acid hydrazide.HCl (MPBH), Succinimidyl-(4-vinylsulfonyl)benzoate (SVSB), dithiobis-maleimidoethane (DTME), 1,4-bis-maleimidobutane (BMB), 1,4 bismaleimidyl-2,3-dihydroxybutane (BMDB), bis-maleimidohexane (BMH), bis-maleimidoethane (BMOE), sulfosuccinimidyl 4-(N-maleimido-methyl)cyclohexane-1-carboxylate (sulfo-SMCC), sulfosuccinimidyl(4-iodo-acetyl)aminobenzoate (sulfo-SIAB), m-Maleimidobenzoyl-N-hydroxysulfosuccinimide ester (sulfo-MBS), N-(γ-maleimidobutryloxy)sulfosuccinimde ester (sulfo-GMBS), N-(ε-maleimidocaproyloxy)sulfosuccimido ester (sulfo-EMCS), N-(κ-maleimidoundecanoyloxy)sulfosuccinimide ester (sulfo-KMUS) and sulfosuccinimidyl 4-(α-maleimidophenyl)butyrate (sulfo-SMPB) CX1-1, sulfo-Mal (or salt thereof) and PEGₙ-Mal. Preferably, the bifunctional crosslinking reagent is SMCC.

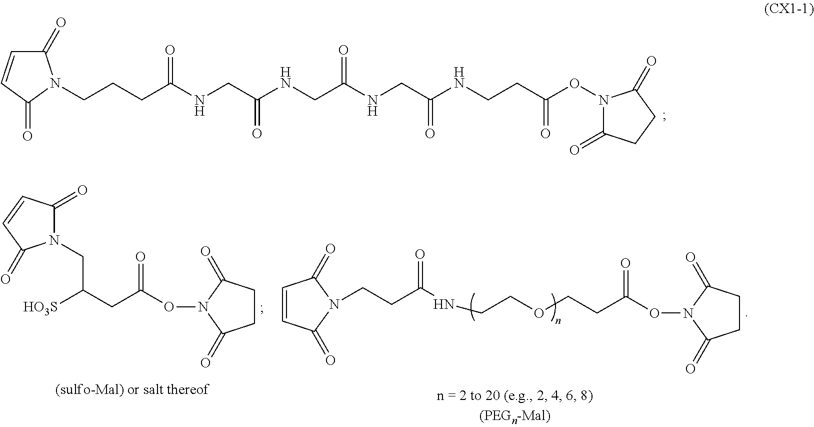

In one embodiment, the linking reagent is a cleavable linker. Examples of suitable cleavable linkers include disulfide linkers, acid labile linkers, photolabile linkers, peptidase labile linkers, and esterase labile linkers. Disulfide containing linkers are linkers cleavable through disulfide exchange, which can occur under physiological conditions. Acid labile linkers are linkers cleavable at acid pH. For example, certain intracellular compartments, such as endosomes and lysosomes, have an acidic pH (pH 4-5), and provide conditions suitable to cleave acid labile linkers. Photo labile linkers are useful at the body surface and in many body cavities that are accessible to light. Furthermore, infrared light can penetrate tissue. Peptidase labile linkers can be used to cleave certain peptides inside or outside cells (see e.g., Trouet et al., Proc. Natl. Acad. Sci. USA, 79: 626-629 (1982), and Umemoto et al., Int. J. Cancer, 43: 677-684 (1989)). In one embodiment, the cleavable linker is cleaved under mild conditions, i.e., conditions within a cell under which the activity of the cytotoxic agent is not affected.

In one embodiment, the cytotoxic agent is linked to a cell-binding agent through a disulfide bond. The linker molecule comprises a reactive chemical group that can react with the cell-binding agent. In one embodiment, the bifunctional crosslinking reagent comprises a reactive moiety that can form an amide bond with a lysine residue of the cell-binding agent. Examples of reactive moieties that can form an amide bond with a lysine residue of a cell-binding agent include carboxylic acid moieties and reactive ester moieties, such as N-succinimidyl ester, N-sulfosuccinimidyl ester, nitrophenyl (e.g., 2 or 4-nitrophenyl) ester, dinitrophenyl (e.g., 2,4-dinitrophenyl) ester, sulfo-tetraflurophenyl (e.g., 4-sulfo-2,3,5,6-tetrafluorophenyl) ester, and pentafluorophenyl ester.

Preferred reactive chemical groups for reaction with the cell-binding agent are N-succinimidyl esters and N-sulfosuccinimidyl esters. Additionally the linker molecule comprises a reactive chemical group, preferably a dithiopyridyl group, that can react with the cytotoxic agent to form a disulfide bond. Bifunctional crosslinking reagents that enable the linkage of the cell-binding agent with the cytotoxic agent via disulfide bonds are known in the art and include, for example, N-succinimidyl 3-(2-pyridyldithio)propionate (SPDP) (see, e.g., Carlsson et al., Biochem. J., 173: 723-737 (1978)), N-succinimidyl 4-(2-pyridyldithio)butanoate (SPDB) or salt thereof (see, e.g., U.S. Pat. No. 4,563,304), N-succinimidyl 4-(2-pyridyldithio)pentanoate (SPP) (see, e.g., CAS Registry number 341498-08-6), and N-succinimidyl-4-(2-pyridyldithio)2-sulfo butanoate (sulfo-SPDB) (see, e.g., U.S. Patent Application Publication No. 2009/0274713). Other bifunctional crosslinking reagents that can be used to introduce disulfide groups are known in the art and are described in U.S. Pat. Nos. 6,913,748, 6,716,821 and U.S. Patent Application Publication Nos. 2009/0274713 and 2010/0129314, all of which are incorporated herein in their entirety by reference.

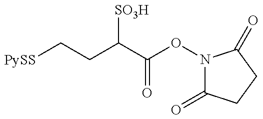

(sulfo-SPDB) or salt thereof

Other crosslinking reagents lacking a sulfur atom that form non-cleavable linkers can also be used in the inventive method. Such linkers can be derived from dicarboxylic acid based moieties. Suitable dicarboxylic acid based moieties include, but are not limited to, α,ω-dicarboxylic acids of the general formula (IX):

HOOC—X₁—Yₙ—Zₘ—COOH (IX),

wherein X is a linear or branched alkyl, alkenyl, or alkynyl group having 2 to 20 carbon atoms, Y is a cycloalkyl or cycloalkenyl group bearing 3 to 10 carbon atoms, Z is a substituted or unsubstituted aromatic group bearing 6 to 10 carbon atoms, or a substituted or unsubstituted heterocyclic group wherein the hetero atom is selected from N, O or S, and wherein l, m, and n are each 0 or 1, provided that l, m, and n are all not zero at the same time.

Many of the non-cleavable linkers disclosed herein are described in detail in U.S. Patent Application Publication No. 2005/0169933 A1.

The following examples further illustrate the invention but, of course, should not be construed as in any way limiting its scope.

### Example 1

This example demonstrates the beneficial effect of adding N-hydroxysuccinimide (NHS) during the modification reaction of a process for preparing a cell-binding agent-cytotoxic agent conjugate. In particular, this example demonstrates that the addition of NHS to the modification reaction has a beneficial effect on the stability of an antibody-maytansinoid conjugate.

Humanized huN901 antibody was reacted with the heterobifunctional crosslinking reagent SMCC and the maytansinoid DM1 using a previously described process (see, e.g., U.S. Pat. No. 5,208,020 and U.S. Patent Application Publication No. 2006/0182750), with or without exogenous NHS added to the modification reaction, in order to make a conjugate with a maytansinoid to antibody ratio (MAR), also known as drug to antibody ratio, of approximately 3.0.

For the previously described process, huN901 (18 mg/mL) first was reacted with SMCC (5.0 fold molar excess relative to the amount of antibody) to form the modified antibody. The modification reaction was performed at 21° C. in 50 mM potassium phosphate, 50 mM potassium chloride, 2 mM EDTA, pH 6.5 and 10% DMA for 150 minutes. A total of about 0.6 mM NHS was released from the combination of the aminolysis reaction of SMCC that leads to attachment of the linker to the antibody and from hydrolysis of SMCC. The modification reaction was quenched with 0.5 M acetic acid to adjust the pH to 5.0, and the modified antibody was purified using a column of Sephadex G25F resin equilibrated and eluted in 20 mM sodium acetate (pH 5.0) containing 2 mM EDTA. After purification, the modified antibody (4 mg/mL) was reacted with the maytansinoid DM1 (4.2 fold molar excess relative to the amount of antibody; 1.3 fold excess relative to the measured amount of linker on the antibody) to form the conjugated antibody. The conjugation reaction was performed at 21° C. in 20 mM sodium acetate buffer (pH 5.0) containing 2 mM EDTA and 5% DMA for approximately 20 hours. The reaction mixture was then purified using a column of Sephadex G25F resin equilibrated and eluted in 10 mM sodium succinate (pH 5.0).

For the process in which exogenous NHS was added, huN901 (18 mg/mL) was reacted with SMCC to form the modified antibody. The modification reaction was performed at 21° C. in 50 mM potassium phosphate, 50 mM potassium chloride, 2 mM EDTA, 10% DMA with the addition of 20 mM, 50 mM, or 100 mM exogenous NHS for 150 minutes. SMCC at a molar excess (relative to the amount of antibody) of 6.7, 10, and 17.5 fold was used for the reactions containing 20 mM, 50 mM, and 100 mM NHS (molar ratio of added NHS to that already present is approximately 25, 42, or 48 fold), respectively. Higher concentrations of SMCC were required for the samples containing higher levels of NHS in order to overcome the inhibitory effect of high concentrations of NHS on the SMCC incorporation. The reaction was quenched with 0.5 M acetic acid to adjust the pH to 5.0, and the modified antibody was purified using a column of Sephadex G25F resin equilibrated and eluted in 20 mM sodium acetate (pH 5.0) containing 2 mM EDTA. After purification, the modified antibody (4 mg/mL) was reacted with the maytansinoid DM1 (3.7 to 4.2 fold molar excess relative to the amount of antibody; 1.3 fold excess relatively to the measured amount of linker on the antibody) to form the conjugated antibody. The conjugation reaction was performed at 21° C. in 20 mM sodium acetate buffer (pH 5.0) containing 2 mM EDTA and 5% DMA for approximately 20 hours. The reaction mixture was then purified using a column of Sephadex G25F resin equilibrated and eluted in 10 mM sodium succinate (pH 5.0).

Conjugates prepared by the processes with or without exogenous NHS in the modification reaction were analyzed for non-reducible species, conjugate monomer, and free maytansinoid.

The non-reducible species level of the conjugates was analyzed by reduced SDS gel electrophoresis. More specifically, peak areas of individual reduced conjugate species (including reduced light chain, reduced heavy chain, cross-linked light-light chains, cross-linked light-heavy chains, etc.) were measured and the non-reducible species level was calculated by the ratio of the sum of areas of non-reducible species to the sum of areas of all species.

The monomer level of the conjugates was analyzed by size exclusion HPLC. More specifically, peak areas of monomer, dimer, aggregates, and low molecular weight species were measured using an absorbance detector set to a wavelength of 252 nm or 280 nm and the monomer level was calculated by the ratio of the monomer area to the total area.

The amount of free maytansinoid present in the conjugate was analyzed by dual column (HiSep and C18 columns) HPLC. More specifically, peak areas of total free maytansinoid species (eluted in the gradient and identified by comparison of elution time with known standards) were measured using an absorbance detector set to a wavelength of 252 nm and the amount of free maytansinoid was calculated using a standard curve generated by the peak areas of known amount of standards.

Conjugate stability with respect to free maytansinoid release and monomer was evaluated by stability testing through storage of the liquid conjugates in 10 mM sodium succinate at 4° C.

As shown in Table 1 below, conjugates manufactured in the presence of exogenous NHS in the modification reaction were superior to conjugates manufactured without exogenous NHS, based on stability with respect to free maytansinoid. Conjugate stability with respect to monomer and non-reducible species was comparable for all samples tested.

TABLE 1
Comparison of key properties of huN901 conjugates (SMCC linker, DM1
maytansinoid) manufactured by the processes with or without
exogenous NHS in the modification reaction

| Exogenous NHS Added | 0 mM NHS | 20 mM NHS | 50 mM NHS | 100 mM NHS |
|---|---|---|---|---|
| Non-reducible species (%) | 8.8 | 7.0 | 7.2 | 7.7 |
| Conjugate monomer | 96.8 | 97.1 | 97.2 | 97.1 |
| (% at t = 0) | | | | |
| Conjugate monomer | 96.8 | 97.0 | 97.0 | 96.9 |
| (% after 10 months at 4° C.) | | | | |
| Free Maytansinoid (% at t = 0) | 0.1 | 0.1 | 0.1 | 0.1 |
| Free Maytansinoid | 7.3 | 2.8 | 1.8 | 1.3 |
| (% after 10.5 months at 4° C.) | | | | |

The results of the experiments presented in this example demonstrate the beneficial effects of adding exogenous NHS during the modification reaction on conjugate stability. In particular, these results confirm that the stability of a huN901-SMCC-DM1 conjugate, as measured by release of free maytansinoid, is significantly enhanced when exogenous NHS is added to the modification reaction.

### Example 2

This example demonstrates the beneficial effect of adding N-hydroxysuccinimide (NHS) during the conjugation reaction of a process for preparing a cell-binding agent-cytotoxic agent conjugate. In particular, this example demonstrates that the addition of NHS to the conjugation reaction has a beneficial effect on the stability of an antibody-maytansinoid conjugate.

Humanized huN901 antibody was reacted with the heterobifunctional crosslinking reagent SMCC and the maytansinoid DM1 using a previously described process (see, e.g., U.S. Pat. No. 5,208,020 and U.S. Patent Application Publication No. 2006/0182750), with or without exogenous N-hydroxysuccinimide (NHS) added into the conjugation reaction, in order to make a conjugate with a maytansinoid to antibody ratio (MAR), also known as drug to antibody ratio, of approximately 3.0.

For this study, huN901 (18 mg/mL) was reacted with SMCC (5.0 fold molar excess relative to the amount of antibody) to form the modified antibody. The modification reaction was performed at 21° C. in 50 mM potassium phosphate, 50 mM potassium chloride, 2 mM EDTA, pH 6.5, and 10% DMA for 150 minutes. The reaction was quenched with 0.5 M acetic acid to adjust the pH to 5.0, and the modified antibody was purified using a column of Sephadex G25F resin equilibrated and eluted in 20 mM sodium acetate (pH 5.0) containing 2 mM EDTA. After purification, the modified antibody (4 mg/mL) was reacted with the maytansinoid DM1 (4.2 fold molar excess relative to the amount of antibody; 1.3 fold excess relative to the measured amount of linker on the antibody) to form the conjugated antibody. The conjugation reaction was performed in 20 mM sodium acetate buffer containing 2 mM EDTA and 5% DMA. The conjugation pH was adjusted to pH 6.5 by adding tribasic sodium phosphate. One conjugation reaction was set up with no NHS added (the previously described process) and three conjugation reactions were set up using with an additional 20 mM, 50 mM, or 100 mM NHS added (the inventive process). The conjugation reactions were held for approximately 20 hours. The reaction mixture was then purified using a column of Sephadex G25F resin equilibrated and eluted in 10 mM sodium succinate (pH 5.0).

Conjugates prepared by the processes with or without exogenous NHS in the conjugation reaction were analyzed for non-reducible species, conjugate monomer, and free maytansinoid using the methods described in Example 1. Conjugate stability with respect to free maytansinoid, non-reducible species, and monomer was evaluated by stability testing through storage at 4° C.

As shown in Table 2 below, conjugates manufactured with exogenous NHS in the conjugation reaction were superior to conjugates manufactured without exogenous NHS, based on free maytansinoid stability. Conjugate stability with respect to non-reducible species and monomer was comparable.

TABLE 2
Comparison of key properties of huN901 conjugates (SMCC linker, DM1
maytansinoid) manufactured by the processes with or without
exogenous NHS in the conjugation reaction

| Exogenous NHS Added | 0 mM NHS | 20 mM NHS | 50 mM NHS | 100 mM NHS |
|---|---|---|---|---|
| Non-reducible species (%) | 8.5 | 8.9 | 8.7 | 7.9 |
| Conjugate monomer | 96.7 | 96.8 | 96.8 | 96.8 |
| (% at t = 0) | | | | |
| Conjugate monomer | 96.8 | 96.8 | 96.8 | 96.9 |
| (% after 10 months at 4° C.) | | | | |
| Free Maytansinoid | 0.1 | ND | ND | ND |
| (% at t = 0) | | | | |
| Free Maytansinoid | 6.7 | 3.1 | 2.8 | 2.4 |
| (% after 10.5 months at 4° C.) | | | | |

ND—not detected

The results of the experiments presented in this example demonstrate the beneficial effects of adding exogenous NHS during the conjugation reaction on conjugate stability. In particular, these results confirm that the stability of a huN901-SMCC-DM1 conjugate, as measured by release of free maytansinoid, is significantly enhanced when exogenous NHS is added to the conjugation reaction.

### Example 3

This example demonstrates the beneficial effect of adding N-hydroxysuccinimide (NHS) to the holding step of a process for preparing a cell-binding agent-cytotoxic agent conjugate. In particular, this example demonstrates that incubating an antibody-maytansinoid conjugate in the presence of exogenous NHS after the conjugation reaction has a beneficial effect on the stability of the antibody-maytansinoid conjugate.

Humanized huN901 antibody was reacted with the heterobifunctional crosslinking reagent SMCC and the maytansinoid DM1, using a previously described process (see, e.g., U.S. Pat. No. 5,208,020 and U.S. Patent Application Publication No. 2006/0182750), with or without exogenous NHS added to the holding step following purification of the conjugation reaction, in order to make a conjugate with a maytansinoid to antibody ratio (MAR), also known as drug to antibody ratio, of approximately 3.0. The purified conjugate was held at different pH values in the absence or presence of exogenous NHS, and then subjected to purification, before conjugate stability was measured.

For the conjugate production, huN901 (18 mg/mL) first was reacted with SMCC (5.0 fold molar excess relative to the amount of antibody) to form the modified antibody. The modification reaction was performed at 21° C. in 50 mM potassium phosphate, 50 mM potassium chloride, 2 mM EDTA, pH 6.5, 10% DMA, for 150 minutes. The reaction was quenched with 0.5 M acetate to adjust the pH to 5.0, and the modified antibody was purified using a column of Sephadex G25F resin equilibrated and eluted in 20 mM sodium acetate (pH 5.0) containing 2 mM EDTA. After purification, the modified antibody (6 mg/mL) was reacted with the maytansinoid DM1 (4.2 fold molar excess relative to the amount of antibody; 1.3 fold excess relative to the measured amount of linker on the antibody) to form the conjugated antibody. The conjugation reaction was performed at 21° C. in 20 mM sodium acetate buffer (pH 5.0) containing 2 mM EDTA and 5% DMA for approximately 20 hours. The reaction mixture was then purified using a column of Sephadex G25F resin equilibrated and eluted in three different buffers: 10 mM sodium succinate pH 5.0; 50 mM sodium phosphate, 2 mM EDTA pH 6.6; or 50 mM sodium phosphate, 2 mM EDTA pH 7.5.

For the holding studies with and without exogenous NHS, purified conjugate at each different pH value was concentrated and diluted with NHS stocks to an antibody concentration of 10 mg/mL and NHS concentrations of 0 mM, 20 mM, 50 mM or 150 mM. The NHS stock solution was pre-adjusted using 1 M NaOH to the same pH as the purified conjugate. The reaction mixtures were held for about 20 hours at ambient temperature and then purified using columns of Sephadex G25F resin equilibrated and eluted in 10 mM sodium succinate pH 5.0.

Conjugates prepared by the processes with or without exogenous NHS in the holding step were analyzed for non-reducible species, conjugate monomer, and free maytansinoid using the methods described in Example 1. Conjugate stability with respect to free maytansinoid release, non-reducible species, and monomer was evaluated by stability testing through storage at 4° C.

As shown in Table 3 below, at all three pH values tested, conjugates prepared with exogenous NHS added to the holding step were superior to those prepared without exogenous NHS added, based on free maytansinoid stability. At a higher pH value for the holding step (pH 6.5 and 7.5), the conjugate stability was improved more significantly when NHS was present during the holding step. There was no significant effect of hold pH value or amount of exogenous NHS added on levels of non-reducible species.

TABLE 3
Comparison of conjugate stability of huN901 conjugates
(SMCC linker, DM1 maytansinoid) manufactured
by the processes with or without exogenous NHS
added during a post conjugation holding step

| | | 0 mM | 20 mM | 50 mM | 150 mM |
|---|---|---|---|---|---|
| | | NHS | NHS | NHS | NHS |
| pH 5.0 | Non-reducible species (%) | 8.9 | 8.3 | 8.4 | 8.6 |
| | Free Maytansinoid | ND | ND | 0.1 | ND |
| | (% at t = 0) | | | | |
| | Free Maytansinoid (% | 6.1 | 5.3 | 4.8 | 5.2 |
| | after 10 months at 4° C.) | | | | |
| pH 6.5 | Non-reducible species (%) | 9.9 | 10.4 | 10.5 | 11.2 |
| | Free Maytansinoid | 0.1 | ND | ND | ND |
| | (% at t = 0) | | | | |
| | Free Maytansinoid (% | 5.1 | 2.5 | 2.0 | 1.6 |
| | after 10 months at 4° C.) | | | | |
| pH 7.5 | Non-reducible species (%) | 10.6 | 10.9 | 9.2 | NT |
| | Free Maytansinoid | ND | ND | ND | ND |
| | (% at t = 0) | | | | |
| | Free Maytansinoid (% | 3.8 | 2.1 | 1.3 | 0.9 |
| | after 10 months at 4° C.) | | | | |

ND—Not detected
NT—Not tested

As shown in Table 4 below, conjugate stability was comparable with respect to the monomer for the conjugates manufactured with or without exogenous NHS added during the holding step.

TABLE 4
Comparison of percent monomer of huN901 conjugates
(SMCC linker, DM1 maytansinoid) manufactured
by the processes with or without exogenous NHS
added during a post conjugation holding step

| | | 0 mM | 20 mM | 50 mM | 150 mM |
|---|---|---|---|---|---|
| | | NHS | NHS | NHS | NHS |
| pH 5.0 | Conjugate monomer | 96.7 | 96.8 | 96.8 | 96.9 |
| | (% at t = 0) | | | | |
| | Conjugate monomer | 96.9 | 96.9 | 96.8 | 96.9 |
| | (% after 10 months | | | | |
| | at 4° C.) | | | | |
| pH 6.5 | Conjugate monomer | 96.6 | 96.7 | 96.7 | 96.7 |
| | (% at t = 0) | | | | |
| | Conjugate monomer | 96.8 | 96.7 | 96.7 | 96.7 |
| | (% after 10 months | | | | |
| | at 4° C.) | | | | |
| pH 7.5 | Conjugate monomer | 96.5 | 96.6 | 96.7 | 96.6 |
| | (% at t = 0) | | | | |
| | Conjugate monomer | 96.7 | 96.6 | 96.5 | 96.6 |
| | (% after 10 months | | | | |
| | at 4° C.) | | | | |

The results of the experiments presented in this example demonstrate the beneficial effects of incubating (i.e., holding) an antibody maytansinoid conjugate in the presence of exogenous NHS on conjugate stability. In particular, these results confirm that the stability of a huN901-SMCC-DM1 conjugate, as measured by release of free maytansinoid, is significantly enhanced when exogenous NHS is added to the holding step after purification of the conjugation reaction.

### Example 4

This example demonstrates the beneficial effect of adding N-hydroxysuccinimide (NHS) during the modification reaction of a process for preparing a cell-binding agent-cytotoxic agent conjugate. In particular, this example demonstrates that the addition of NHS to the modification reaction has a beneficial effect on the stability of an antibody-maytansinoid conjugate.

Humanized huC242 antibody was reacted with the heterobifunctional crosslinking reagent SPDB and the maytansinoid DM4 using a previously described process (see, e.g., U.S. Pat. No. 7,811,572), with or without exogenous NHS added to the modification reaction, in order to make a conjugate with a maytansinoid to antibody ratio (MAR), also known as drug to antibody ratio, of approximately 4.0.

For the previously described process, huC242 (8 mg/mL) first was reacted with SPDB (5.8 fold molar excess relative to the amount of antibody) to form the modified antibody. The modification reaction was performed at room temperature in 50 mM potassium phosphate, 50 mM sodium chloride, 2 mM EDTA, pH 6.5, and 5% DMA for 150 minutes. A total of about 0.3 mM NHS was released from the combination of the aminolysis reaction of SPDB that leads to attachment of the linker to the antibody and from hydrolysis of SPDB. The modified antibody was not purified before the conjugation reaction. Instead, the unpurified modified antibody was diluted to 4.0 mg/mL and was reacted with the maytansinoid DM4 (9.8-fold molar excess relative to the amount of antibody) to form the conjugated antibody. The conjugation reaction was performed at room temperature in 50 mM potassium phosphate, 50 mM sodium chloride, 2 mM EDTA, pH 6.5 containing 5% DMA for approximately 19 hours. The reaction mixture was then purified using a column of Sephadex G25F resin equilibrated and eluted in phosphate buffer pH 6.5.

For the process in which exogenous NHS was added, huC242 (8 mg/mL) was reacted with SPDB at room temperature in 50 mM potassium phosphate, 50 mM sodium chloride, 2 mM EDTA, 5% DMA, and either 1.6 mM, 3.1 mM, or 6.3 mM exogenous NHS (molar ratio of added NHS to that already present is approximately 5, 10, or 20, respectively) for 150 minutes. Un-purified modified antibody was diluted to 4 mg/mL and reacted with the maytansinoid DM4 (9.8 fold molar excess relative to the amount of antibody) to form the conjugated antibody. The conjugation reaction was performed at room temperature in 50 mM potassium phosphate, 50 mM sodium chloride, 2 mM EDTA, pH 6.5 containing 5% DMA for approximately 19 hours. The reaction mixture was then purified using a column of Sephadex G25F resin equilibrated and eluted in phosphate buffer pH 6.5.

Conjugates prepared by the processes with or without exogenous NHS in the modification reaction were analyzed for conjugate monomer and free maytansinoid.

The monomer level of the conjugates was analyzed by size exclusion HPLC. More specifically, peak areas of monomer, dimer, aggregates, and low molecular weight species were measured using an absorbance detector set to a wavelength of 280 nm and the monomer level was calculated by the ratio of the monomer area to the total area.

The amount of free maytansinoid present in the conjugate was analyzed by HiSep HPLC. More specifically, peak areas of total free maytansinoid species (eluted in the gradient and identified by comparison of elution time with known standards) were measured using an absorbance detector set to a wavelength of 252 nm, the peak area of the bound maytansinoid was calculated from the flow through peak using A252 absorbance, and the percentage of free maytansinoid was calculated by dividing the peak areas of total free maytansinoid with the total peak area of free and bound maytansinoid.

Conjugate stability with respect to free maytansinoid release was evaluated by stability testing through storage at 4° C.

As shown in Table 5 below, conjugates manufactured with exogenous NHS added in the modification reaction were superior to conjugates manufactured without exogenous NHS, based on free maytansinoid levels observed at t=0, as well as after storage for 59 weeks at 4° C. Conjugate stability with respect to monomer was comparable for all samples tested.

TABLE 5
Comparison of key properties of huC242 conjugates (SPDB linker,
DM4 maytansinoid) manufactured by the processes with or
without additional NHS in the modification reaction

| Exogenous | 0 mM | 1.6 mM | 3.1 mM | 6.3 mM |
|---|---|---|---|---|
| NHS Added | NHS | NHS | NHS | NHS |
| Conjugate monomer | 95.1 | 94.9 | 95.3 | 95.7 |
| (% at t = 0) | | | | |
| Free Maytansinoid | 1.1 | 0.8 | 0.7 | 0.5 |
| (% at t = 0) | | | | |
| Free Maytansinoid | 2.8 | 2.1 | 1.8 | 1.4 |
| (% at 59 weeks | | | | |
| at 4° C.) | | | | |

The results of the experiments presented in this example demonstrate the beneficial effects of adding exogenous NHS during the modification reaction on conjugate stability. In particular, these results confirm that the stability of a huC242-SPDB-DM4 conjugate, as measured by release of free maytansinoid, is significantly enhanced when exogenous NHS is added to the modification reaction.

### Example 5

This example demonstrates the beneficial effect of adding N-hydroxysuccinimide (NHS) during the conjugation reaction of a process for preparing a cell-binding agent-cytotoxic agent conjugate. In particular, this example demonstrates that the addition of NHS to the conjugation reaction has a beneficial effect on the stability of an antibody-maytansinoid conjugate.

Humanized huDS6 antibody was reacted with the heterobifunctional crosslinking reagent SPDB and the maytansinoid DM4 using a previously described process (see, e.g., U.S. Pat. No. 5,208,020 and U.S. Patent Application Publication No. 2006/0182750), with or without exogenous N-hydroxysuccinimide (NHS) added to the conjugation reaction, in order to make a conjugate with a maytansinoid to antibody ratio (MAR), also known as drug to antibody ratio, of approximately 3.5.

For this study, huDS6 (10 mg/mL) was reacted with SPDB (4.3 fold molar excess relative to the amount of antibody) to form the modified antibody. The modification reaction was performed at room temperature in 50 mM potassium phosphate, 100 mM sodium chloride, and 5% DMA at pH 7.5 for 60 minutes. The modified antibody was purified using a column of Sephadex G25F resin equilibrated and eluted in 50 mM potassium phosphate, 100 mM sodium chloride, pH 7.5. After purification, the modified antibody (4 mg/mL) was reacted with the maytansinoid DM4 (6.8 fold molar excess relative to the amount of antibody; 1.7 fold excess relatively to the measured amount of linker on the antibody) to form the conjugated antibody. The conjugation reaction was performed in 50 mM potassium phosphate, 100 mM sodium chloride with 5% DMA at pH 7.5. Two conjugation reactions were set up using either no added NHS (the previously described process), or with 0.3 mM NHS added (the inventive process). The conjugation reactions were held for approximately 21 hours at room temperature. The reaction mixture was then purified using a column of Sephadex G25F resin equilibrated and eluted in phosphate buffer pH 6.5.

Conjugates prepared by the processes with or without exogenous NHS in the conjugation reaction were analyzed for conjugate monomer and free maytansinoid using HPLC as described in Example 4. Conjugate stability with respect to free maytansinoid release was evaluated by stability testing through storage at 4° C.

As shown in Table 6 below, conjugate manufactured with as little as 0.3 mM exogenous NHS added to the conjugation reaction was superior to conjugate manufactured without exogenous NHS based on stability with respect to free maytansinoid.

TABLE 6
Comparison of key properties of huDS6-SPDB-DM4 manufactured by the
processes with or without exogenous NHS in the conjugation reaction

| Exogenous NHS Added | 0 mM NHS | 0.3 mM NHS |
|---|---|---|
| Conjugate monomer (%, 1 day at 4° C.) | 95.6 | 95.3 |
| Free Maytansinoid (%, 1 day at 4° C.) | 1.1 | 0.4 |
| Free Maytansinoid (%, 24 weeks at 4° C.) | 2.8 | 1.0 |

The results of the experiments presented in this example demonstrate the beneficial effects of adding exogenous NHS during the conjugation reaction on conjugate stability. In particular, these results confirm that the stability of a huDS6-SPDB-DM4 conjugate, as measured by release of free maytansinoid, is significantly enhanced when exogenous NHS is added to the conjugation reaction.

### Example 6

This example demonstrates the beneficial effect of adding N-hydroxysuccinimide (NHS) during the modification reaction of a process for preparing a cell-binding agent-cytotoxic agent conjugate. In particular, this example demonstrates that the addition of NHS to the modification reaction has a beneficial effect on the stability of an antibody-maytansinoid conjugate.

Humanized huDS6 antibody was reacted with the heterobifunctional crosslinking reagent SPDB and the maytansinoid DM4 using a previously described process (see, e.g., U.S. Pat. No. 5,208,020 and U.S. Patent Application Publication No. 2006/0182750), with or without exogenous NHS added to the modification reaction, in order to make a conjugate with a maytansinoid to antibody ratio (MAR), also known as drug to antibody ratio, of approximately 3.0.

For the previously described process, huDS6 (10 mg/mL) first was reacted with SPDB (4.3 fold molar excess relative to the amount of antibody) to form the modified antibody. The modification reaction was performed at room temperature in 50 mM potassium phosphate, 100 mM sodium chloride, pH 7.5, and 5% DMA for 15 minutes. A total of about 0.3 mM NHS was released from the combination of the aminolysis reaction of SPDB that leads to attachment of the linker to the antibody and from hydrolysis of SPDB. The modified antibody was purified using a column of Sephadex G25F resin equilibrated and eluted in 50 mM potassium phosphate, 100 mM sodium chloride, pH 7.5. After purification, the modified antibody (4 mg/mL) was reacted with the maytansinoid DM4 (7.1 fold molar excess relative to the amount of antibody; 1.7 fold excess relative to the measured amount of linker on the antibody) to form the conjugated antibody. The conjugation reaction was performed at room temperature in 50 mM potassium phosphate, 100 mM sodium chloride, pH 7.5, and 5% DMA for approximately 20 hours. The reaction mixture was then purified using a column of Sephadex G25F resin equilibrated and eluted in phosphate buffer pH 6.5 (Buffer B).

For the process in which exogenous NHS was added, huDS6 (10 mg/mL) was reacted with SPDB for 15 minutes at room temperature in 50 mM potassium phosphate, 100 mM sodium chloride, pH 7.5, 5% DMA, with an additional 3 mM NHS (molar ratio of added NHS to that already present is approximately 10 fold). The modified antibody was purified using a column of Sephadex G25F resin equilibrated and eluted in 50 mM potassium phosphate, 100 mM sodium chloride, pH 7.5. After purification, the modified antibody (4 mg/mL) was reacted with the maytansinoid DM4 (6.6 fold molar excess relative to the amount of antibody; 1.7 fold excess relative to the measured amount of linker on the antibody) to form the conjugated antibody. The conjugation reaction was performed at room temperature in 50 mM potassium phosphate, 100 mM sodium chloride, pH 7.5, and 5% DMA for approximately 20 hours. The reaction mixture was then purified using a column of Sephadex G25F resin equilibrated and eluted in phosphate buffer pH 6.5 (Buffer B).

Conjugates prepared by the processes with or without exogenous NHS in the modification reaction were analyzed for conjugate monomer and free maytansinoid using HPLC as described in Example 4. Conjugate stability with respect to free maytansinoid release was evaluated by stability testing through storage at 4° C.

As shown in Table 7 below, conjugate manufactured with exogenous NHS added in the modification reaction was superior to conjugates manufactured without exogenous NHS, based on stability with respect to free maytansinoid.

TABLE 7
Comparison of key properties of huDS6-SPDB-DM4 manufactured by the
processes with or without exogenous NHS in the modification reaction

| Exogenous NHS Added | 0 mM NHS | 3 mM NHS |
|---|---|---|
| Conjugate monomer (% at t = 0) | 95.7 | 95.4 |
| Free Maytansinoid (% at t = 0) | 0.8 | 0.2 |
| Free Maytansinoid (% after 62 weeks at 4° C.) | 5.6 | 1.9 |

The results of the experiments presented in this example demonstrate the beneficial effects of adding exogenous NHS during the modification reaction on conjugate stability. In particular, these results confirm that the stability of a huDS6-SPDB-DM4 conjugate, as measured by release of free maytansinoid, is significantly enhanced when exogenous NHS is added to the modification reaction

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A process for preparing a cell-binding agent having a linker bound thereto, which process comprises contacting a cell-binding agent with a bifunctional crosslinking reagent in the presence of exogenous N-hydroxysuccinimide (NHS) to covalently attach a linker to the cell-binding agent and thereby prepare a mixture comprising cell-binding agents having linkers bound thereto.

2. A process for preparing a cell-binding agent-cytotoxic agent conjugate comprising a cell-binding agent chemically coupled to a cytotoxic agent through a linker, which process comprises:
(a) contacting a cell-binding agent with a bifunctional crosslinking reagent to covalently attach a linker to the cell-binding agent and thereby prepare a first mixture comprising cell-binding agents having linkers bound thereto,
(b) subjecting the first mixture to tangential flow filtration, selective precipitation, non-adsorptive chromatography, adsorptive filtration, adsorptive chromatography, or a combination thereof and thereby prepare a purified first mixture of cell-binding agents having linkers bound thereto,
(c) conjugating a cytotoxic agent to the cell-binding agents having linkers bound thereto in the purified first mixture by reacting the cell-binding agents having linkers bound thereto with a cytotoxic agent to prepare a second mixture comprising (i) the cell-binding agent-cytotoxic agent conjugate comprising the cell-binding agent chemically coupled to the cytotoxic agent through the linker, (ii) free cytotoxic agent, and (iii) reaction by-products, and
(d) subjecting the second mixture to tangential flow filtration, selective precipitation, non-adsorptive chromatography, adsorptive filtration, adsorptive chromatography, or a combination thereof to purify the cell-binding agent-cytotoxic agent conjugate from the other components of the second mixture and thereby prepare a purified second mixture of the cell-binding agent-cytotoxic agent conjugate,
wherein exogenous N-hydroxysuccinimide is added during or after step (a) and prior to step (c).

3. The process of claim 2, wherein the contacting in step (a) is carried out in the presence of exogenous N-hydroxysuccinimide.

4. The process of claim 2, further comprising holding the first mixture after step (a) in the presence of exogenous N-hydroxysuccinmide.

5. (canceled)

6. (canceled)

7. The process of claim 2, wherein the exogenous N-hydroxysuccinmide is added in step (b).

8. The process of claim 2, further comprising holding the purified first mixture after step (b) in the presence of exogenous N-hydroxysuccinmide.

9.-21. (canceled)

22. A process for preparing a cell-binding agent-cytotoxic agent conjugate comprising a cell-binding agent chemically coupled to a cytotoxic agent through a linker, which process comprises:
(a) contacting a cell-binding agent with a bifunctional crosslinking reagent to covalently attach a linker to the cell-binding agent and thereby prepare a first mixture comprising cell-binding agents having linkers bound thereto,
(b) conjugating a cytotoxic agent to the cell-binding agents having linkers bound thereto in the first mixture by reacting the cell-binding agents having linkers bound thereto with a cytotoxic agent to prepare a second mixture comprising (i) the cell-binding agent-cytotoxic agent conjugate comprising the cell-binding agent coupled to the cytotoxic agent through the linker, (ii) free cytotoxic agent, and (iii) reaction by-products, and
(c) subjecting the second mixture to tangential flow filtration, selective precipitation, non-adsorptive chromatography, adsorptive filtration, adsorptive chromatography, or a combination thereof, to purify the cell-binding agent-cytotoxic agent conjugate from the other components of the second mixture and thereby prepare a purified second mixture of the cell-binding agent-cytotoxic agent conjugate,
wherein exogenous N-hydroxysuccinimide is added during of after step (a) and prior to step (b).

23. The process of claim 22, wherein the contacting in step (a) is carried out in the presence of exogenous N-hydroxysuccinimide.

24. The process of claim 22, further comprising holding the first mixture after step (a) in the presence of exogenous N-hydroxysuccinimide.

25.-35. (canceled)

36. A process for preparing a cell-binding agent-cytotoxic agent conjugate comprising a cell-binding agent chemically coupled to a cytotoxic agent through a linker, which process comprises:
(a) contacting a cell-binding agent with a cytotoxic agent-linker compound comprising a cytotoxic agent chemically coupled to a linker to covalently attach the cytotoxic agent-linker compound to the cell-binding agent and thereby prepare a mixture comprising the cell-binding agent-cytotoxic agent conjugate comprising the cell-binding agent chemically coupled to the cytotoxic agent through the linker and
(b) subjecting the mixture comprising the cell-binding agent-cytotoxic agent conjugate to tangential flow filtration, selective precipitation, non-adsorptive chromatography, adsorptive filtration, adsorptive chromatography or a combination thereof to purify the conjugate, wherein exogenous N-hydroxysuccinimide is added during or after step (a) and prior to step (b).

37. The process of claim 36, wherein the contacting in step (a) is carried out in the presence of exogenous N-hydroxysuccinimide.

38. The process of claim 36, further comprising holding the mixture after step (a) in the presence of exogenous N-hydroxysuccinimide.

39.-46. (canceled)

47. A process for preparing a cell-binding agent-cytotoxic agent conjugate comprising a cell-binding agent chemically coupled to a cytotoxic agent through a linker, which process comprises:
(a) contacting a cell-binding agent with a bifunctional crosslinking reagent to covalently attach a linker to the cell-binding agent and thereby prepare a first mixture comprising cell-binding agents having linkers bound thereto,
(b) subjecting the first mixture to tangential flow filtration, selective precipitation, non-adsorptive chromatography, adsorptive filtration, adsorptive chromatography, or a combination thereof and thereby prepare a purified first mixture of cell-binding agents having linkers bound thereto,
(c) conjugating a cytotoxic agent to the cell-binding agents having linkers bound thereto in the purified first mixture by reacting the cell-binding agents having linkers bound thereto with a cytotoxic agent in the presence of exogenous N-hydroxysuccinimide to prepare a second mixture comprising (i) the cell-binding agent-cytotoxic agent conjugate comprising the cell-binding agent coupled to the cytotoxic agent through the linker, (ii) free cytotoxic agent, and (iii) reaction by-products, and
(d) subjecting the second mixture to tangential flow filtration, selective precipitation, non-adsorptive chromatography, adsorptive filtration, adsorptive chromatography, or a combination thereof to purify the cell-binding agent-cytotoxic agent conjugate from the other components of the second mixture and thereby prepare a purified second mixture of cell-binding agent-cytotoxic agent conjugate.

48.-58. (canceled)

59. A process for preparing a cell-binding agent-cytotoxic agent conjugate comprising a cell-binding agent chemically coupled to a cytotoxic agent through a linker, which process comprises:
(a) contacting a cell-binding agent with a bifunctional crosslinking reagent to covalently attach a linker to the cell-binding agent and thereby prepare a first mixture comprising cell-binding agents having linkers bound thereto,
(b) conjugating a cytotoxic agent to the cell-binding agents having linkers bound thereto in the first mixture by reacting the cell-binding agents having linkers bound thereto with a cytotoxic agent in the presence of exogenous N-hydroxysuccinimide to prepare a second mixture comprising (i) the cell-binding agent-cytotoxic agent conjugate comprising cell-binding agent chemically coupled through the linker to the cytotoxic agent, (ii) free cytotoxic agent, and (iii) reaction by-products, and
(c) subjecting the second mixture to tangential flow filtration, selective precipitation, non-adsorptive chromatography, adsorptive filtration, adsorptive chromatography, or a combination thereof, to purify the cell-binding agent-cytotoxic agent conjugate from the other components of the second mixture and thereby prepare a purified second mixture of the cell-binding agent-cytotoxic agent conjugate.

60.-68. (canceled)

69. A process for preparing a cell-binding agent-cytotoxic agent conjugate comprising a cell-binding agent chemically coupled to a cytotoxic agent through a linker, which process comprises:
(a) contacting a cell-binding agent with a bifunctional crosslinking reagent to covalently attach a linker to the cell-binding agent and thereby prepare a first mixture comprising cell-binding agents having linkers bound thereto,
(b) subjecting the first mixture to tangential flow filtration, selective precipitation, non-adsorptive chromatography, adsorptive filtration, adsorptive chromatography, or a combination thereof and thereby prepare a purified first mixture of cell-binding agents having linkers bound thereto,
(c) conjugating a cytotoxic agent to the cell-binding agents having linkers bound thereto in the purified first mixture by reacting the cell-binding agents having linkers bound thereto with a cytotoxic agent to prepare a second mixture comprising (i) the cell-binding agent-cytotoxic agent conjugate comprising the cell-binding agent chemically coupled through the linker to the cytotoxic agent, (ii) free cytotoxic agent, and (iii) reaction by-products,
(d) incubating the second mixture in the presence of exogenous N-hydroxysuccinimide; and
(e) subjecting the second mixture after step (d) to tangential flow filtration, selective precipitation, non-adsorptive chromatography, adsorptive filtration, adsorptive chromatography, or a combination thereof to purify the cell-binding agent-cytotoxic agent conjugate from the other components of the second mixture and thereby prepare a purified second mixture of cell-binding agents chemically coupled through the linkers to the cytotoxic agent.

70.-87. (canceled)

88. A process for preparing a conjugate comprising a cell-binding agent chemically coupled to a cytotoxic agent through a linker, which process comprises:
(a) contacting a cell-binding agent with a bifunctional crosslinking reagent to covalently attach a linker to the cell-binding agent and thereby prepare a first mixture comprising cell-binding agents having linkers bound thereto,
(b) conjugating a cytotoxic agent to the cell-binding agents having linkers bound thereto in the first mixture by reacting the cell-binding agents having linkers bound thereto with a cytotoxic agent to prepare a second mixture comprising (i) the cell-binding agent-cytotoxic agent conjugate comprising the cell-binding agent chemically coupled through the linker to the cytotoxic agent, (ii) free cytotoxic agent, and (iii) reaction by-products,
(c) incubating the second mixture in the presence of exogenous N-hydroxysuccinimide; and
(d) subjecting the second mixture after step (c) to tangential flow filtration, selective precipitation, non-adsorptive chromatography, adsorptive filtration, adsorptive chromatography, or a combination thereof, to purify the cell-binding agent-cytotoxic agent conjugate from the other components of the second mixture and thereby prepare a purified second mixture of the cell-binding agent-cytotoxic agent conjugate.

89.-103. (canceled)

104. A process for preparing a cell-binding agent-cytotoxic agent conjugate comprising a cell-binding agent chemically coupled to a cytotoxic agent through a linker, which process comprises:
(a) contacting a cell-binding agent with a cytotoxic agent to form a first mixture comprising the cell-binding agent and the cytotoxic agent, then contacting the first mixture with a bifunctional crosslinking reagent comprising a linker, in a solution having a pH of about 4 to about 9, to provide a second mixture comprising (i) the cell-binding agent cytotoxic agent conjugate comprising the cell-binding agent chemically coupled through the linker to the cytotoxic agent, (ii) free cytotoxic agent, and (iii) reaction by-products;
(b) incubating the second mixture in the presence of exogenous N-hydroxysuccinimide; and
(c) subjecting the second mixture after step (b) to tangential flow filtration, selective precipitation, non-adsorptive chromatography, adsorptive filtration, adsorptive chromatography, or a combination thereof, to purify the cell-binding agent-cytotoxic agent conjugate from the other components of the second mixture and thereby prepare a purified second mixture of the cell-binding agent-cytotoxic agent conjugate.

105.-116. (canceled)

117. A process for preparing a cell-binding agent-cytotoxic agent conjugate comprising a cell-binding agent chemically coupled to a cytotoxic agent through a linker, which process comprises:
(a) contacting a cell-binding agent with a cytotoxic agent in the presence of exogenous N-hydroxysuccinimide to form a first mixture comprising the cell-binding agent and the cytotoxic agent, then contacting the first mixture with a bifunctional crosslinking reagent comprising a linker, in a solution having a pH of about 4 to about 9, to provide a second mixture comprising (i) the cell-binding agent cytotoxic agent conjugate comprising the cell-binding agent chemically coupled through the linker to the cytotoxic agent, (ii) free cytotoxic agent, and (iii) reaction by-products; and
(b) subjecting the second mixture to tangential flow filtration, selective precipitation, non-adsorptive chromatography, adsorptive filtration, adsorptive chromatography, or a combination thereof, to purify the cell-binding agent-cytotoxic agent conjugate from the other components of the second mixture and thereby prepare a purified second mixture of the cell-binding agent-cytotoxic agent conjugate.

118. A process for preparing a cell-binding agent-cytotoxic agent conjugate comprising a cell-binding agent chemically coupled to a cytotoxic agent through a linker, which process comprises:
(a) contacting a cell-binding agent with a cytotoxic agent to form a first mixture comprising the cell-binding agent and the cytotoxic agent, then contacting the first mixture with a bifunctional crosslinking reagent comprising a linker in the presence of exogenous N-hydroxysuccinimide, in a solution having a pH of about 4 to about 9, to provide a second mixture comprising (i) the cell-binding agent cytotoxic agent conjugate comprising the cell-binding agent chemically coupled through the linker to the cytotoxic agent, (ii) free cytotoxic agent, and (iii) reaction by-products; and
(b) subjecting the second mixture to tangential flow filtration, selective precipitation, non-adsorptive chromatography, adsorptive filtration, adsorptive chromatography, or a combination thereof, to purify the cell-binding agent-cytotoxic agent conjugate from the other components of the second mixture and thereby prepare a purified second mixture of the cell-binding agent-cytotoxic agent conjugate.

119.-123. (canceled)

124. A process for preparing a cell-binding agent-cytotoxic agent conjugate comprising a cell-binding agent chemically coupled to a cytotoxic agent through a linker, which process comprises:
(a) contacting a cell-binding agent with a cytotoxic agent-linker compound comprising a cytotoxic agent chemically coupled to a linker to covalently attach the cytotoxic agent-linker compound to the cell-binding agent and thereby prepare a mixture comprising the cell-binding agent-cytotoxic agent conjugate comprising the cell-binding agent chemically coupled to the cytotoxic agent through the linker;
(b) incubating the mixture of step (a) in the presence of exogenous N-hydroxysuccinimide; and
(c) subjecting the mixture after step (b) to tangential flow filtration, selective precipitation, non-adsorptive chromatography, adsorptive filtration, adsorptive chromatography, or a combination thereof, to purify the cell binding agent-cytotoxic agent conjugate from the other components of the mixture and thereby prepare a purified mixture of the cell binding agent-cytotoxic agent conjugate.

125.-159. (canceled)
